(19) United States
(12) Patent Application Publication
(10) Pub. No.: US 2022/0033829 A1
(43) Pub. Date: Feb. 3, 2022

(54) PEPTIDE BARCODES FOR CORRELATING NUCLEIC ACID-GUIDED NUCLEASE OR NICKASE FUSION EDITING AND PROTEIN TRANSLATION IN A POPULATION OF CELLS

(71) Applicant: Inscripta, Inc., Boulder, CO (US)

(72) Inventors: Tyson Shepherd, Boulder, CO (US); Andrew Garst, Boulder, CO (US); Daniel Held, Boulder, CO (US); Eric Abbate, Boulder, CO (US)

(21) Appl. No.: 17/387,780

(22) Filed: Jul. 28, 2021

Related U.S. Application Data
(60) Provisional application No. 63/058,545, filed on Jul. 30, 2020.

(51) Int. Cl. C12N 15/63; C12N 9/22; C12N 15/11

(52) U.S. Cl. CPC C12N 15/63; C12N 2310/20; C12N 15/111; C12N 9/22

(57) ABSTRACT

The present disclosure relates to compositions, methods, modules and automated integrated instrumentation for using sets of short, curated peptide barcodes to track nucleic acid-guided edits and the translated proteins that result from the edits as well as to create in vitro pathways.

### RELATED CASES

This application claims priority to U.S. Ser. No. 63/058,545, filed 30 Jul. 2020, entitled “Peptide Barcodes for Correlating Nucleic Acid-Guided Nuclease Editing and Protein Translation in a Population of Cells”, which is incorporated herein in its entirety.

### FIELD OF THE INVENTION

The present disclosure relates to compositions, methods, modules and automated integrated instrumentation for using sets of short, curated peptide barcodes to track nucleic acid-guided edits and the translated proteins that result from the edits.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate that the articles and methods referenced herein do not constitute prior art under the applicable statutory provisions.

The ability to make precise, targeted changes to the genome of living cells has been a long-standing goal in biomedical research and development. Recently, various nucleases have been identified that allow for manipulation of gene sequences; hence gene function. The nucleases include nucleic acid-guided nucleases, which enable researchers to generate permanent edits in live cells. In addition to editing a nucleotide sequence, it is of interest to uniquely and easily identify an edited target sequence and any resulting change in the level of translation of proteins in the edited cells.

There is thus a need in the art of nucleic acid-guided nuclease editing for improved methods, compositions, modules and automated, integrated instruments to assess and improve tracking genome-wide edits with protein production, where the protein production can be quantified easily. The present disclosure addresses this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure relates to methods, compositions, modules and automated multi-module cell processing instruments that allow one to perform nucleic acid-guided nuclease editing, assess and track genome-wide edits with protein production and easily quantify the resulting protein production via mass spectrometry. The present compositions and methods are applicable to assessing the impact of a genomic edit on protein production; creating two-hybrid systems; creating purified cell-free pathways and translation systems in “one pot”; and engineering proteins for production and/or secretion.

Thus, there is provided a method for editing a population of live cells with a library of editing vectors comprising rationally-designed editing cassettes, measuring levels of one or more proteins from the live cells and correlating the edits in the live cells to protein quantities comprising: designing and synthesizing a library of editing cassettes wherein each editing cassette comprises a gRNA and a repair template, wherein the repair template encodes a barcode construct comprising a peptide barcode, an affinity tag and a protease cleavage site; inserting the library of editing cassettes into vector backbones to form a library of editing vectors; transferring the library of editing vectors into a first receptacle; providing cells to be edited in a second receptacle; growing the cells to be edited in a growth module; transferring the cells to be edited from the growth module to a cell concentration module; concentrating and rendering electrocompetent the cells to be edited in the cell concentration module; introducing the library of editing vectors into the electrocompetent cells in a transformation module to produce transformed cells; allowing editing to take place in the transformed cells to produce edited cells, wherein the edited cells transcribe and translate edited proteins comprising each of the peptide barcode, the affinity tag and the protease cleavage site; pooling and lysing the edited cells; performing protease cleavage at the protease cleavage site in the edited proteins to cleave the affinity tag and peptide barcodes from a rest of the edited proteins; isolating the peptide barcodes via the affinity tags; identifying and quantitating the peptide barcodes; and correlating the edits with the quantity of peptide barcodes; wherein the first receptacle, second receptacle, third receptacle, growth module, cell concentration module, transformation module and editing module are all part of a stand-alone automated multi-module cell processing instrument.

In some aspects, the barcode comprises between three to twenty amino acids, and in some aspects, the barcode comprises between five to fifteen amino acids, and in yet other aspects, the barcode comprises between seven to twelve amino acids.

In some aspects, the barcode construct is inserted 3′ to a cellular protein start codon and 5′ to the remainder of the coding sequence for the cellular protein, and in some configurations of this aspect, the barcode construct comprises 5′ to 3′ the peptide barcode, the affinity tag, and the protease cleavage site. In other configuration of this aspect, the barcode construct comprises 5′ to 3′ the affinity tag, the peptide barcode, and the protease cleavage site. In some aspects, the barcode construct is inserted 5′ to a cellular protein stop codon, and in some configurations of this aspect, the barcode construct comprises 5′ to 3′ the protease cleavage site, the peptide barcode, and the affinity tag. In yet another configuration of this aspect, the barcode construct comprises 5′ to 3′ the protease cleavage site, the affinity tag, and the peptide barcode. In some configurations, an affinity tag is 3′ of a start codon, followed by a cleavage site, followed by the peptide barcode, followed by a second cleavage site of the same or different protease. In some configurations, the same applies for a C-terminal tag, where 5′ of the stop codon would be a cleavage site, a peptide barcode, a second cleavage site, and an affinity tag, followed by a stop codon.

In some aspects of this method embodiment, the affinity tag is a histidine tag, and in some configurations, the histidine tag is a His6 tag. In yet other aspects, the affinity tag is a Glutathione S-Transferase tag, or a calmodulin-binding tag.

In some aspects, the protease cleavage site is a TEV protease cleavage site and in some aspects, the protease cleavage site is a thrombin protease cleavage site.

Some embodiments provide an editing cassette comprising a gRNA and a repair template, wherein the repair template comprises a coding sequence for a peptide barcode, a coding sequence for an affinity tag, a coding sequence for a protease cleavage site and homology arms complementary to sequences 5′ or 3′ to a cellular protein coding sequence. Some embodiments of provide a library of editing cassettes each comprising a gRNA and a repair template, wherein the repair template comprises a coding sequence for a peptide barcode, a coding sequence for an affinity tag, a coding sequence for a protease cleavage site and homology arms complementary to sequences 5′ or 3′ to a cellular protein coding sequence.

These aspects and other features and advantages of the invention are described below in more detail.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1A is a simple process diagram for performing nucleic acid-guided nuclease editing in a population of cells to add a peptide barcode to correlate an edited target sequence to resulting protein levels. FIG. 1B is a simplified diagram of a construct for a coding sequence for a cellular protein after nucleic acid-guided editing takes place to add a peptide barcode /affinity tag/protease cleavage site (a “barcode construct”) to the 5′ end of the protein coding sequence. FIG. 1C is a simplified diagram of a construct for a coding sequence for a cellular protein after nucleic acid-guided editing takes place to add a peptide barcode /affinity tag/protease cleavage site (a “barcode construct”) to the 3′ end of the protein coding sequence. FIG. 1D is an exemplary coding sequence for the N-terminal methionine of the tagged cellular protein, an affinity tag, a peptide barcode and a TEV protease cleavage site.

FIGS. 2A-2C depict three different views of an exemplary automated multi-module cell processing instrument for performing nucleic acid-guided nuclease editing.

FIG. 3A depicts one embodiment of a rotating growth vial for use with the cell growth module described herein and in relation to FIGS. 3B-3D. FIG. 3B illustrates a perspective view of one embodiment of a rotating growth vial in a cell growth module housing. FIG. 3C depicts a cut-away view of the cell growth module from FIG. 3B. FIG. 3D illustrates the cell growth module of FIG. 3B coupled to LED, detector, and temperature regulating components.

FIG. 4A depicts retentate (top) and permeate (bottom) members for use in a tangential flow filtration module (e.g., cell growth and/or concentration module), as well as the retentate and permeate members assembled into a tangential flow assembly (bottom). FIG. 4B depicts two side perspective views of a reservoir assembly of a tangential flow filtration module. FIGS. 4C-4E depict an exemplary top, with fluidic and pneumatic ports and gasket suitable for the reservoir assemblies shown in FIG. 4B.

FIGS. 5A and 5B depict the structure and components of an embodiment of a reagent cartridge. FIG. 5C is a top perspective view of one embodiment of an exemplary flow-through electroporation device that may be part of a reagent cartridge. FIG. 5D depicts a bottom perspective view of one embodiment of an exemplary flow-through electroporation device that may be part of a reagent cartridge. FIGS. 5E-5G depict a top perspective view, a top view of a cross section, and a side perspective view of a cross section of an FTEP device useful in a multi-module automated cell processing instrument such as that shown in FIGS. 2A-2C.

FIG. 6A depicts a simplified graphic of a workflow for singulating, editing, normalizing, and back-end analysis of cells in a solid wall device. FIGS. 6B-6D depict an embodiment of a solid wall isolation incubation and normalization (SWIIN) module. FIG. 6E depicts the embodiment of the SWIIN module in FIGS. 6B-6D further comprising a heater and a heated cover.

FIGS. 7A-7G depict various components of an embodiment of a bioreactor useful for growing and transducing mammalian cells by the methods described herein. FIG. 7H-1 and 7H-2 depict an exemplary fluidic diagram for the bioreactor described in relation to FIGS. 7A-7G. FIG. 7I depicts an exemplary control system block diagram for the bioreactor described in relation to FIGS. 7A-7G.

It should be understood that the drawings are not necessarily to scale, and that like reference numbers refer to like features.

### DETAILED DESCRIPTION

All of the functionalities described in connection with one embodiment of the methods, devices or instruments described herein are intended to be applicable to the additional embodiments of the methods, devices and instruments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of molecular biology (including recombinant techniques), cell biology, biochemistry, and genetic engineering technology, which are within the skill of those who practice in the art. Such conventional techniques and descriptions can be found in standard laboratory manuals such as Green and Sambrook, Molecular Cloning: A Laboratory Manual. 4th, ed., Cold Spring Harbor Laboratory Press, Cold Spring Harbor, N.Y., (2014); Current Protocols in Molecular Biology, Ausubel, et al. eds., (2017); Neumann, et al., Electroporation and Electrofusion in Cell Biology, Plenum Press, New York, 1989; Chang, et al., Guide to Electroporation and Electrofusion, Academic Press, Calif. (1992); Viral Vectors (Kaplift & Loewy, eds., Academic Press 1995), all of which are herein incorporated in their entirety by reference for all purposes. For mammalian/stem cell culture and methods see, e.g., Basic Cell Culture Protocols, Fourth Ed. (Helgason & Miller, eds., Humana Press 2005); Culture of Animal Cells, Seventh Ed. (Freshney, ed., Humana Press 2016); Microfluidic Cell Culture, Second Ed. (Borenstein, Vandon, Tao & Charest, eds., Elsevier Press 2018); Human Cell Culture (Hughes, ed., Humana Press 2011); 3D Cell Culture (Koledova, ed., Humana Press 2017); Cell and Tissue Culture: Laboratory Procedures in Biotechnology (Doyle & Griffiths, eds., John Wiley & Sons 1998); Essential Stem Cell Methods, (Lanza & Klimanskaya, eds., Academic Press 2011); Essentials of Stem Cell Biology, Third Ed., (Lanza & Atala, eds., Academic Press 2013); and Handbook of Stem Cells, (Atala & Lanza, eds., Academic Press 2012), all of which are herein incorporated in their entirety by reference for all purposes. Nucleic acid-guided nuclease techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “a cell” refers to one or more cells, and reference to “the system” includes reference to equivalent steps, methods and devices known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

As used herein, the terms “amplify” or “amplification” and their derivatives, refer to any operation or process whereby at least a portion of a nucleic acid molecule is replicated or copied into at least one additional nucleic acid molecule. The additional nucleic acid molecule may include a sequence that is substantially identical or substantially complementary to at least a portion of the template nucleic acid molecule. The template nucleic acid molecule can be single-stranded or double-stranded, and the additional nucleic acid molecule can be independently single-stranded or double-stranded. Amplification may include linear or exponential replication of a nucleic acid molecule. In certain embodiments, amplification can be achieved using isothermal conditions; in other embodiments, amplification may include thermocycling. In certain embodiments, the amplification is a multiplex amplification and includes the simultaneous amplification of a plurality of target sequences in a single reaction or process. In certain embodiments, “amplification” includes amplification of at least a portion of DNA and RNA based nucleic acids. The amplification reaction(s) can include any of the amplification processes known to those of ordinary skill in the art. In certain embodiments, the amplification reaction(s) includes methods such as polymerase chain reaction (PCR), ligase chain reaction (LCR), or other methods.

The term “complementary” as used herein refers to Watson-Crick base pairing between nucleotides and specifically refers to nucleotides hydrogen-bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TAGCTG-3′.

The term DNA “control sequence” or “control element” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and—for some components—translated in an appropriate host cell.

The terms “editing cassette”, “CREATE cassette”, “CREATE editing cassette”, “CREATE fusion editing cassette” or “CFE editing cassette” refers to a nucleic acid molecule comprising a coding sequence for transcription of a guide nucleic acid or gRNA covalently linked to a coding sequence for transcription of a repair template.

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclease or nickase fusion enzyme.

“Homology” or “identity” or “similarity” refers to sequence similarity between two peptides or, more often in the context of the present disclosure, between two nucleic acid molecules. The term “homologous region” or “homology arm” refers to a region on the repair template with a certain degree of homology with the target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

As used herein, the term “nickase fusion” refers to a nucleic acid-guided nickase-(or nucleic acid-guided nuclease or CRISPR nuclease) that has been engineered to act as a nickase rather than a nuclease (e.g., the nickase portion of the fusion functions as a nickase as opposed to a nuclease that initiates double-stranded DNA breaks), where the nickase is fused to a reverse transcriptase, which is an enzyme used to generate cDNA from an RNA template. For information regarding nickase-RT fusions see, e.g., U.S. Pat. No. 10,689,669 and U.S. Ser. No. 16/740,421.

“Nucleic acid-guided editing components” refers to one, some, or all of a nucleic acid-guided nuclease or nickase fusion enzyme, a guide nucleic acid and a repair template.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

As used herein, the terms “protein” and “polypeptide” are used interchangeably. Proteins may or may not be made up entirely of amino acids.

As used herein, the term “peptide barcodes” refers to unique genetically-encoded peptide sequences designed to enable identification of a genetic edit with which a protein is associated. The term “barcode construct” refers to coding sequences for a peptide barcode, affinity tag and peptide cleavage site.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA transcribed by any class of any RNA polymerase I, II or III. Promoters may be constitutive or inducible.

As used herein the term “repair template” refers to nucleic acid that is designed to introduce a DNA sequence modification (insertion, deletion, substitution) into a locus by homologous recombination using nucleic acid-guided nucleases or a nucleic acid that serves as a template (including a desired edit) to be incorporated into target DNA by reverse transcriptase in a nickase fusion editing system.

As used herein the term “selectable marker” refers to a gene introduced into a cell, which confers a trait suitable for artificial selection. General use selectable markers are well-known to those of ordinary skill in the art. Drug selectable markers such as ampicillin/carbenicillin, kanamycin, chloramphenicol, nourseothricin N-acetyl transferase, erythromycin, tetracycline, gentamicin, bleomycin, streptomycin, puromycin, hygromycin, blasticidin, and G418 may be employed. In other embodiments, selectable markers include, but are not limited to human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34+cells); CD24 cell surface antigen in hematopoietic stem cells; human CAD gene to confer resistance to N-phosphonacetyl-L-aspartate (PALA); human multi-drug resistance-1 (MDR-1; P-glycoprotein surface protein selectable by increased drug resistance or enriched by FACS); human CD25 (IL-2a; detectable by Mab-FITC); Methylguanine-DNA methyltransferase (MGMT; selectable by carmustine); rhamnose; and Cytidine deaminase (CD; selectable by Ara-C). “Selective medium” as used herein refers to cell growth medium to which has been added a chemical compound or biological moiety that selects for or against selectable markers.

The term “specifically binds” as used herein includes an interaction between two molecules, e.g., an engineered peptide antigen and a binding target, with a binding affinity represented by a dissociation constant of about 10⁻⁷M, about 10⁻⁸ M, about 10⁻⁹ M, about 10⁻¹⁰M, about 10⁻¹¹M, about 10⁻¹²M, about 10⁻¹³M, about 10⁻¹⁴M or about 10⁻¹⁵M.

The terms “target genomic DNA sequence”, “cellular target sequence”, “target sequence”, “target genome”, “target cellular locus” or “genomic target locus” refer to any locus in vitro or in vivo, or in a nucleic acid (e.g., genome or episome) of a cell or population of cells, in which a change of at least one nucleotide is desired using a nucleic acid-guided nuclease editing system. The target sequence can be a genomic locus or extrachromosomal locus. The term “edited target sequence” or “edited locus” refers to a target genomic sequence or target sequence after editing has been performed, where the edited target sequence comprises the desired edit.

The terms “transformation”, “transfection” and “transduction” are used interchangeably herein to refer to the process of introducing exogenous DNA into cells.

The term “variant” may refer to a polypeptide or polynucleotide that differs from a reference polypeptide or polynucleotide but retains essential properties. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A variant of a polypeptide may be a conservatively modified variant. A substituted or inserted amino acid residue may or may not be one encoded by the genetic code (e.g., a non-natural amino acid). A variant of a polypeptide may be naturally occurring, such as an allelic variant, or it may be a variant that is not known to occur naturally.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, synthetic chromosomes, and the like. In some embodiments of the present methods, two vectors—an engine vector, comprising the coding sequences for a nuclease, and an editing vector, comprising the gRNA sequence and the repair template sequence—are used. In alternative embodiments, all editing components, including the nuclease, gRNA sequence, and repair template sequence are all on the same vector (e.g., a combined editing/engine vector).

### Nuclease-Directed Genome Editing Generally

The compositions, methods, modules and instruments described herein are employed to allow one to perform nucleic acid nuclease- or nickase fusion-directed genome editing to introduce desired edits to a population of live cells as well as to uniquely identify the consequences of the edits. Specifically, the compositions, methods, modules and integrated instruments presented herein—in addition to editing nucleotide sequences in a rational and explicit manner—utilize peptide barcodes which allow one to uniquely and easily correlate an edited target sequence with the resulting profile of one or more proteins.

A nucleic acid-guided nuclease or nickase fusion complexed with an appropriate synthetic guide nucleic acid in a cell can cut the genome of the cell at a desired location. The guide nucleic acid helps the nucleic acid-guided nuclease or nickase fusion recognize and cut the DNA at a specific target sequence. By manipulating the nucleotide sequence of the guide nucleic acid, the nucleic acid-guided nuclease or nickase fusion enzyme may be programmed to target any DNA sequence for cleavage as long as an appropriate protospacer adjacent motif (PAM) is nearby. In certain aspects, the nucleic acid-guided nuclease or nickase fusion editing system may use two separate guide nucleic acid molecules that combine to function as a guide nucleic acid, e.g., a CRISPR RNA (crRNA) and trans-activating CRISPR RNA (tracrRNA). In other aspects and preferably, the guide nucleic acid is a single guide nucleic acid construct that includes both 1) a guide sequence capable of hybridizing to a genomic target locus, and 2) a scaffold sequence capable of interacting or complexing with a nucleic acid-guided nuclease or nickase fusion.

In general, a guide nucleic acid (e.g., gRNA) complexes with a compatible nucleic acid-guided nuclease or nickase fusion and can then hybridize with a target sequence, thereby directing the nuclease or nickase fusion to the target sequence. A guide nucleic acid can be DNA or RNA; alternatively, a guide nucleic acid may comprise both DNA and RNA. In some embodiments, a guide nucleic acid may comprise modified or non-naturally occurring nucleotides. In cases where the guide nucleic acid comprises RNA, the gRNA may be encoded by a DNA sequence on a polynucleotide molecule such as a plasmid, linear construct, or the coding sequence may and preferably does reside within an editing cassette. Methods and compositions for designing and synthesizing editing cassettes are described in U.S. Pat. Nos. 10,240,167; 10,266,849; 9,982,278; 10,351,877; 10,364,442; 10,435,715; 10,465,207; and 10,669,559; and U.S. Ser. Nos. 16/773,618; and 16/773,712, all of which are incorporated by reference herein.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease or nickase fusion to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

In general, to generate an edit in the target sequence, the gRNA/nuclease or nickase fusion complex binds to a target sequence as determined by the guide RNA, and the nuclease or nickase fusion recognizes a protospacer adjacent motif (PAM) sequence adjacent to the target sequence. The target sequence can be any polynucleotide endogenous or exogenous to the cell, or in vitro. For example, the target sequence can be a polynucleotide residing in the nucleus of the cell. A target sequence can be a sequence encoding a gene product (e.g., a protein) or a non-coding sequence (e.g., a regulatory polynucleotide, an intron, a PAM, a control sequence, or “junk” DNA).

The guide nucleic acid may be and preferably is part of an editing cassette that encodes the repair template that targets a cellular target sequence. Alternatively, the guide nucleic acid may not be part of the editing cassette and instead may be encoded on the editing vector backbone. For example, a sequence coding for a guide nucleic acid can be assembled or inserted into a vector backbone first, followed by insertion of the repair template in, e.g., an editing cassette. In other cases, the repair template in, e.g., an editing cassette can be inserted or assembled into a vector backbone first, followed by insertion of the sequence coding for the guide nucleic acid. Preferably, the sequence encoding the guide nucleic acid and the repair template are located together in a rationally-designed editing cassette and are simultaneously inserted or assembled via gap repair into a linear plasmid or vector backbone to create an editing vector.

The target sequence is associated with a proto-spacer mutation (PAM), which is a short nucleotide sequence recognized by the gRNA/nuclease or nickase fusion complex. The precise preferred PAM sequence and length requirements for different nucleic acid-guided nucleases or nickase fusions vary; however, PAMs typically are 2-7 base-pair sequences adjacent or in proximity to the target sequence and, depending on the nuclease or nickase fusion, can be 5′ or 3′ to the target sequence. Engineering of the PAM-interacting domain of a nucleic acid-guided nuclease or nickase fusion may allow for alteration of PAM specificity, improve target site recognition fidelity, decrease target site recognition fidelity, or increase the versatility of a nucleic acid-guided nuclease or nickase fusion.

In most embodiments, genome editing of a cellular target sequence both introduces a desired DNA change to a cellular target sequence (an “intended” edit—here, the at least one intended comprises the insertion of a barcode construct), e.g., the genomic DNA of a cell, and removes, mutates, or renders inactive a proto-spacer mutation (PAM) region in the cellular target sequence (an “immunizing edit”) thereby rendering the target site immune to further nuclease or nickase fusion binding. Rendering the PAM at the cellular target sequence inactive precludes additional editing of the cell genome at that cellular target sequence, e.g., upon subsequent exposure to a nucleic acid-guided nuclease or nickase fusion complexed with a synthetic guide nucleic acid in later rounds of editing. Thus, cells having the desired cellular target sequence edit and an altered PAM can be selected for by using a nucleic acid-guided nuclease or nickase fusion complexed with a synthetic guide nucleic acid complementary to the cellular target sequence. Cells that did not undergo the first editing event will be cut rendering a double-stranded DNA break, and thus will not continue to be viable. The cells containing the desired cellular target sequence edit and PAM alteration will not be cut, as these edited cells no longer contain the necessary PAM site and will continue to grow and propagate.

As for the nuclease or nickase fusion component of the nucleic acid-guided nuclease or nickase fusion editing system, a polynucleotide sequence encoding the nucleic acid-guided nuclease or nickase fusion can be codon optimized for expression in particular cell types, such as bacterial, yeast, and mammalian cells. The choice of the nucleic acid-guided nuclease or nickase fusion to be employed depends on many factors, such as what type of edit is to be made in the target sequence and whether an appropriate PAM is located close to the desired target sequence. Nucleases of use (or nickase fusions may be derived from) in the methods described herein include but are not limited to Cas 9, Cas 12/CpfI, MAD2, or MAD7 or other MADzymes.

Another component of the nucleic acid-guided nuclease or nickase fusion system is the repair template comprising homology to the cellular target sequence. For the present methods and compositions, the repair template is on the same vector and in the same editing cassette as the guide nucleic acid and is under the control of the same promoter as the editing gRNA (that is, a single promoter driving the transcription of both the editing gRNA and the repair template). The repair template is designed to serve as a template for homologous recombination with a cellular target sequence nicked or cleaved by the nucleic acid-guided nuclease or nickase fusion as a part of the gRNA/nuclease or nickase fusion complex. In the present case, at least one repair template comprises the barcode construct; that is, coding sequences for the peptide barcode, the affinity tag and the protease cleavage site. A repair template polynucleotide may be of any suitable length, such as about or more than about 20, 25, 50, 75, 100, 150, 200, 500, or 1000 nucleotides in length, and up to 2, 3, 4, 5, 6, 7, 8, 9, 10, 11, 12, 13 and up to 20 kb in length if combined with a dual gRNA architecture as described in U.S. Ser. No. 16/275,465, filed 14 Feb. 2019. In certain preferred aspects, the repair template can be provided as an oligonucleotide of between 20-300 nucleotides, more preferably between 50-250 nucleotides. The repair template comprises two regions that are complementary to a portion of the cellular target sequence (e.g., homology arms) flanking the mutation or difference between the repair template and the cellular target sequence. When optimally aligned, the repair template overlaps with (is complementary to) the cellular target sequence by, e.g., about 20, 25, 30, 35, 40, 50, 60, 70, 80, 90 or more nucleotides. The repair template comprises at least one mutation or alteration compared to the cellular target sequence, such as an insertion, deletion, modification, or any combination thereof compared to the cellular target sequence.

As described in relation to the gRNA, the repair template is provided as part of a rationally-designed editing cassette, which is inserted into an editing plasmid backbone (in yeast, preferably a linear plasmid backbone) where the editing plasmid backbone may comprise a promoter to drive transcription of the editing gRNA and the repair template when the editing cassette is inserted into the editing plasmid backbone. Moreover, there may be more than one, e.g., two, three, four, or more editing gRNA/repair template rationally-designed editing cassettes inserted into an editing vector; alternatively, a single rationally-designed editing cassette may comprise two to several editing gRNA/repair template pairs, where each editing gRNA is under the control of separate different promoters, separate like promoters, or where all gRNAs/repair template pairs are under the control of a single promoter. In some embodiments the promoter driving transcription of the editing gRNA and the repair template (or driving more than one editing gRNA/repair template pair) is optionally an inducible promoter.

In addition to the repair template, an editing cassette may comprise one or more primer binding sites. The primer binding sites are used to amplify the editing cassette by using oligonucleotide primers as described infra and may be biotinylated or otherwise labeled. In some embodiments, the editing cassettes comprise a collection or library editing gRNAs and of repair templates representing, e.g., gene-wide or genome-wide libraries of editing gRNAs and repair templates. The library of editing cassettes is cloned into vector backbones where, e.g., each different repair template is associated with a different barcode. Also, in preferred embodiments, an editing vector or plasmid encoding components of the nucleic acid-guided nuclease or nickase fusion system further encodes a nucleic acid-guided nuclease or nickase fusion comprising one or more nuclear localization sequences (NLSs), such as about or more than about 1, 2, 3, 4, 5, 6, 7, 8, 9, 10, or more NLSs, particularly as an element of the nuclease or nickase fusion sequence. In some embodiments, the engineered nuclease or nickase fusion comprises NLSs at or near the amino-terminus, NLSs at or near the carboxy-terminus, or a combination.

Making Rational Genome Edits and Using Peptide Barcodes to Determine the Consequences of the Genome Edits and/or to Generate Proteins of Interest in a “One Pot” Reaction

The present disclosure is drawn to methods, compositions, modules and automated, integrated instruments that edit live cells to create a change in genomic nucleic acids and—in addition to editing nucleotide sequences in a rational and explicit manner—utilize peptide barcodes which allow one to uniquely and easily correlate an edited target sequence and the resulting profile of one or more proteins. The peptide barcodes are covalently linked to both an affinity tag and a protease cleavage site; collectively, a “barcode construct.” The affinity tag allows for rapid isolation of the peptide barcodes and the protease cleavage site is used to separate the peptide barcode/affinity tag pair from the remaining portion of the protein, if desired. The peptide barcodes are designed to be easily detectable and easily distinguishable from one another using mass spectrometry.

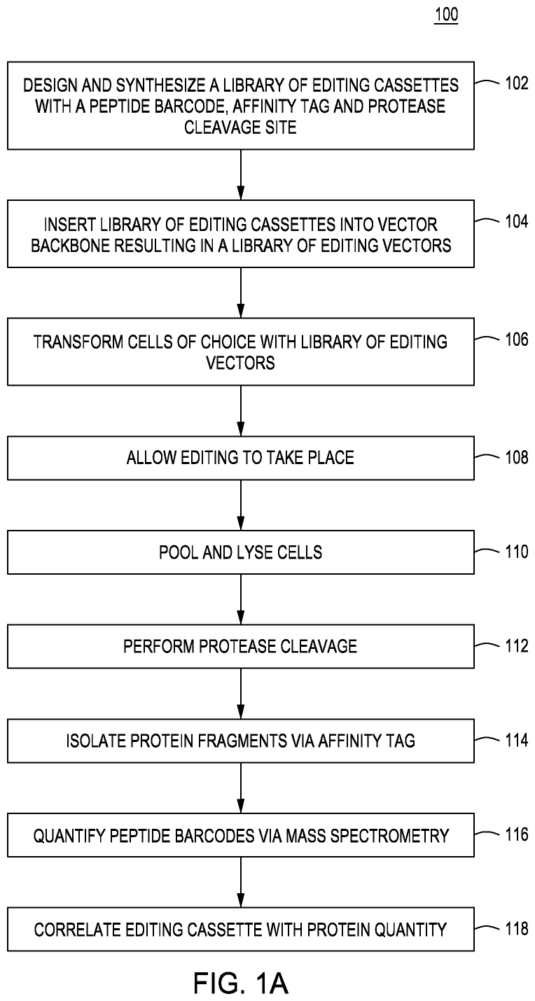

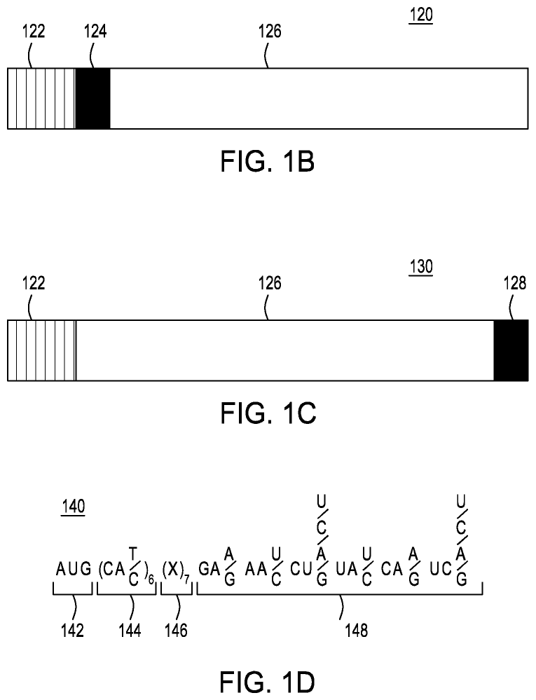

FIG. 1A is a simple process diagram for performing nucleic acid-guided nuclease or nickase fusion editing in a population of cells while adding a barcode construct comprising a peptide barcode to correlate edits with a resulting level of translation of one or more proteins. In a first step 102 of method 100, a library of editing cassettes comprising paired gRNAs and repair templates is designed and synthesized. The repair template portion of the editing cassettes each comprise coding sequences for a peptide barcode, an affinity tag and a protease cleavage site (collectively, a “barcode construct”), which is engineered to be inserted 5′ or 3′ of a genomic coding sequence for a cellular protein (or open reading frame of a protein). Each peptide barcode is unique for the protein with which it is associated upon editing (e.g., each editing cassette will code for a different peptide barcode); however, in most embodiments the coding sequences for the affinity tags in each editing cassette will code for the same affinity tag so that all protein barcodes may be purified in one step. The editing cassette may also comprise additional sequences such as one or more priming sequences that can be used to amplify the editing cassette. The various components of the editing cassettes are described in more detail infra in relation to FIGS. 1B and 1C.

Once designed and synthesized 102, the library of editing cassettes is amplified, purified and inserted 104 into a vector backbone—which in some embodiments may already comprise a coding sequence for the nuclease or nickase fusion—to produce a library of editing vectors. Alternatively, the coding sequence for the nuclease or nickase fusion may be located on another vector that may be transformed into the cells before, at the same time as or after the editing vectors are transformed into the cells. In yet other alternatives, the coding sequence for the nuclease or nickase fusion may be integrated into the cellular genome or the nuclease or nickase fusion may be delivered to the cell as a protein. The vectors chosen for the methods herein will vary depending on the type of cells being edited and analyzed, where the vectors include, e.g., plasmids, BACs, YACs, viral vectors and synthetic chromosomes.

The cells of interest useful in the methods herein are any cells, including bacterial, yeast and animal (including mammalian) cells. Before being transformed by the editing vectors, the cells are often grown in culture for several passages. Cell culture is the process by which cells are grown under controlled conditions, almost always outside the cell's natural environment. For bacterial and yeast cells, the cells are typically grown in a defined medium in bulk culture. For mammalian cells, culture conditions typically vary somewhat for each cell type but generally include a medium and additives that supply essential nutrients such as amino acids, carbohydrates, vitamins, minerals, growth factors, hormones, and gases such as, e.g., O₂ and CO₂. In addition to providing nutrients, the medium typically regulates the physio-chemical environment via a pH buffer and most cells are grown at 37° C. Many mammalian cells require or prefer a surface or artificial substrate on which to grow (e.g., adherent cells), whereas other cells such as hematopoietic cells and some adherent cells can be grown in or adapted to grow in suspension. Adherent cells often are grown in 2D monolayer cultures in petri dishes or flasks, but some adherent cells can grow in suspension cultures to higher density than would be possible in 2D cultures. “Passages” generally refers to transferring a small number of cells to a fresh substrate with fresh medium, or, in the case of suspension cultures, transferring a small volume of the culture to a larger volume of medium.

The cells of choice are provided and are transformed with the library of editing vectors 106. The library of editing vectors comprises vector backbones each “carrying” at least one editing cassette, wherein at least one editing cassette comprises the coding sequences for a peptide barcode, an affinity tag and a protease cleavage site engineered to be inserted 5′ or 3′ of a genomic coding sequence for a cellular protein. Again, each peptide barcode is unique for the cellular protein (that is, the coding sequence for the cellular protein into which it is inserted) and each editing cassette will code for a different peptide barcode; however, the coding sequences for the affinity tags in each editing cassette in most embodiments will code for the same affinity tag. The library of editing cassettes may have tens, hundreds, thousands, tens of thousands or more different editing cassettes, dependent upon the number of unique peptide barcodes available.

Additionally, the editing vectors may comprise in addition to the editing cassette comprising the barcode construct, one or more additional editing cassettes configured to target other regions of the cellular genome. The one or more additional editing cassettes may be configured to create promoter or terminator swaps, SNP swaps, insertions, deletions, etc.

As used herein, transformation is intended to generically include a variety of art-recognized techniques for introducing an exogenous nucleic acid sequence (e.g., an engine and/or editing vector) into a target cell, and the term “transformation” as used herein includes all transformation and transfection techniques. Such methods include, but are not limited to, electroporation, lipofection, optoporation, injection, microprecipitation, microinjection, liposomes, particle bombardment, sonoporation, laser-induced potation, bead transfection, calcium phosphate or calcium chloride co-precipitation, or DEAE-dextran-mediated transfection. Cells can also be prepared for vector uptake using, e.g., a sucrose, sorbitol or glycerol wash. Additionally, hybrid techniques that exploit the capabilities of mechanical and chemical transfection methods can be used, e.g, magnetofection, a transfection methodology that combines chemical transfection with mechanical methods. In another example, cationic lipids may be deployed in combination with gene guns or electroporators. Suitable materials and methods for transforming or transfecting target cells can be found, e.g., in Green and Sambrook, Molecular Cloning: A Laboratory Manual, 4th, ed., Cold Spring Harbor Laboratory Press, Cold Spring Harbor, N.Y., 2014).

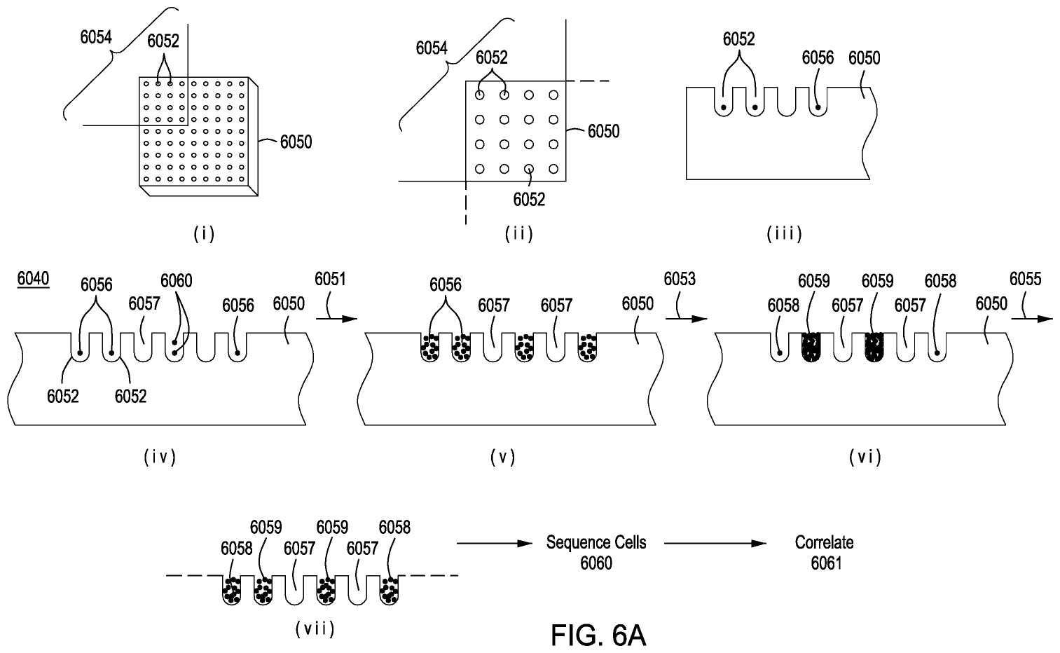

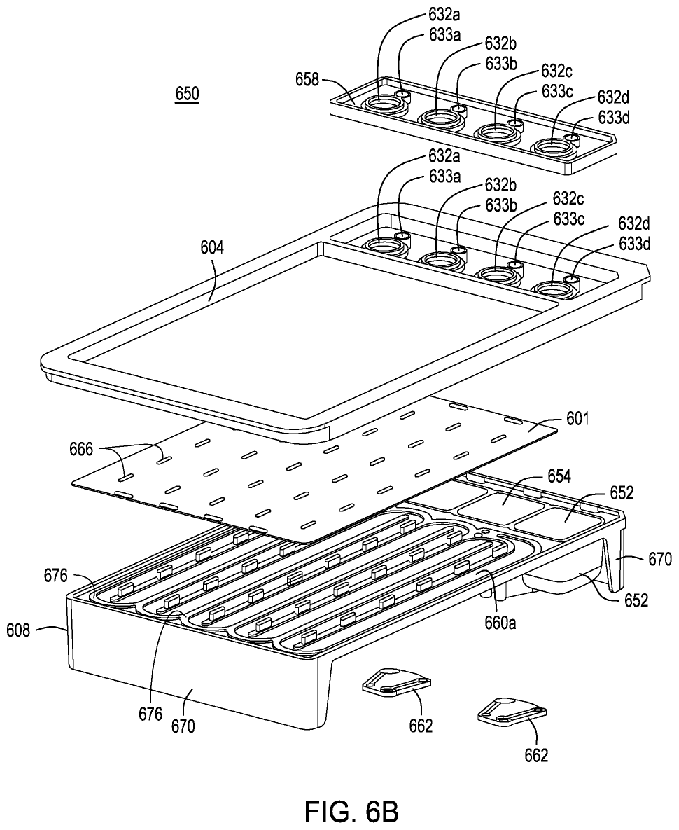

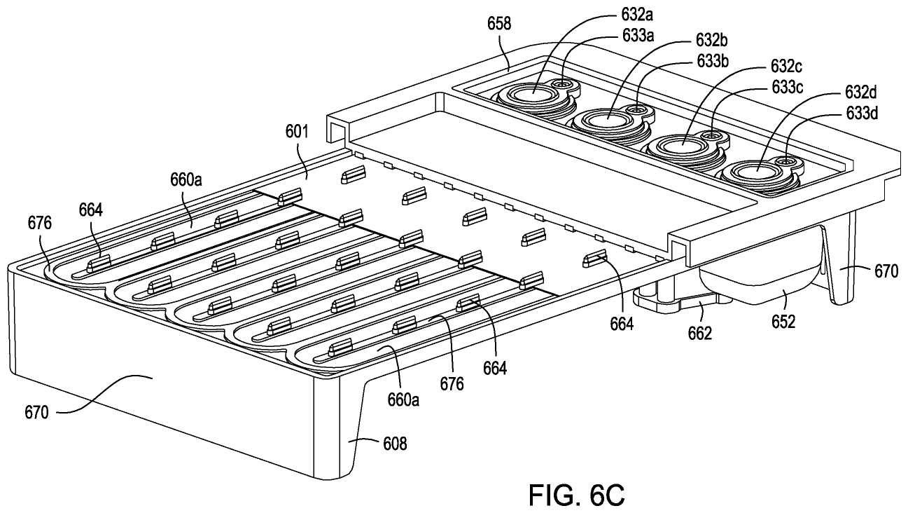

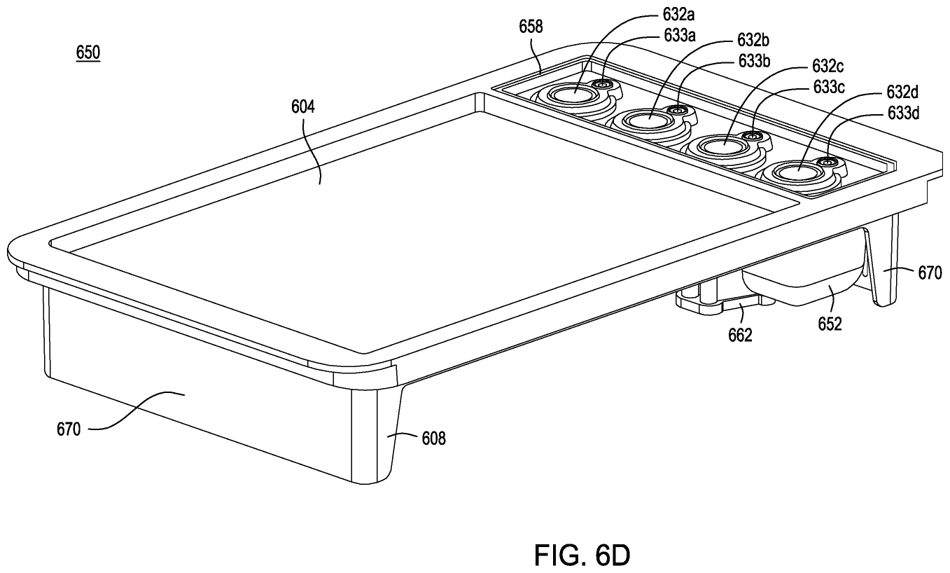

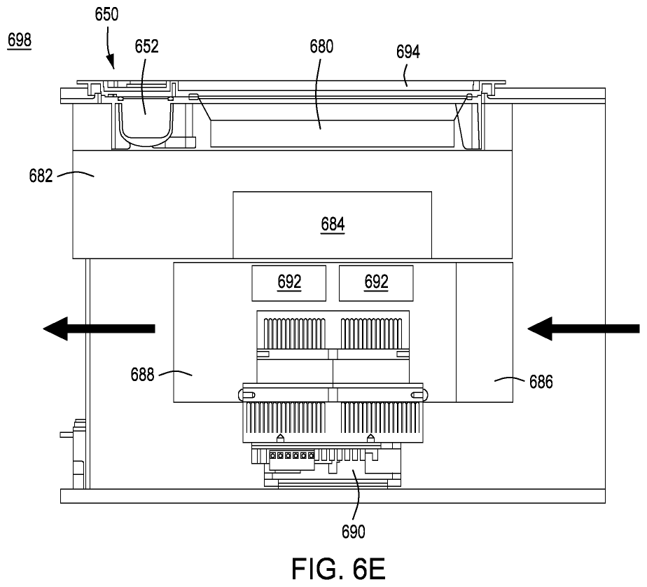

Once transformed 106, the cells are allowed to recover and selection optionally is performed to select for cells transformed with the editing vector, which most often comprises a selectable marker. At a next step 108, editing is allowed to take place. If one or both components of the editing machinery (e.g., editing cassette and nuclease or nickase fusion enzyme) is under the control of an inducible promoter, conditions are provided to induce editing. It none of the components of the editing machinery are under the control of an inducible promoter, editing proceeds immediately after transformation. During the editing process, many cells will die due to double-strand breaks in the genome that are a consequence of the editing process. Methods and a module for neutralizing the effects of the editing process on the transformed cell population are described infra in relation to FIGS. 6A-6E. Of the cells that do survive editing and continue to grow, the cells will transcribe and translate cellular proteins, including the protein(s) edited and “tagged” by the peptide barcodes and affinity tags.

After editing takes place and after recovery and growth for 1-4 hours, or typically 8, 10 or 14 hours in rich medium and optional antibiotic selection at 15-37° C., the cells are pooled then lysed 110. After the cells are pooled and lysed 110, the cell lysate optionally can be centrifuged to remove solid cellular debris, or subjected to detergent lysis, and then the entire cell lysate from the pooled population of cells can be subjected to binding with a binding partner of the affinity tags that have been inserted into (e.g., edited into) the cellular proteins (this step is not shown in this FIG. 1A). The process of removing solid cellular debris and performing an initial isolation step for the affinity-tagged cellular proteins is performed to “clean up” the cell lysate prior to protease cleavage 112, but again, these steps are optional.

If the cell lysate is not “cleaned up”, once the cells are pooled, grown, and lysed 110 protease cleavage is performed 112 by using a protease to cleave the affinity-tagged cellular proteins into two portions: 1) the peptide barcode/affinity tag portion, and 2) the remainder of the cellular protein. Protease cleavage is described in more detail infra in relation to FIG. 1B. Once protease cleavage has taken place 112, the peptide barcode/affinity tag portions of the cellular proteins are isolated/purified using the affinity tags 114. As noted above, affinity tags are peptide sequences engineered to be “grafted” onto a cellular protein—in the present instance, coding sequences for the affinity tags—along with a coding sequence for a peptide barcode—are edited into a coding sequence for a cellular protein. Here, affinity tags are used to purify the peptide barcodes from the crude cellular extract using an affinity technique such as affinity chromatography. Various affinity tags are described in more detail infra in relation to FIG. 1B.

After purification/isolation of the peptide barcodes 114, the peptide barcodes are quantified by mass spectrometry 116. Because the peptide barcodes are unique for the edit made and for the cellular protein edited, the edits made in the cell population can be correlated 118 with the quantity of the resulting proteins without sequencing.

FIG. 1B is a simplified diagram of an edited construct 120 after adding a peptide barcode/affinity tag/protease cleavage site to the 5′ end of the protein coding sequence. Element 122 is a control element, e.g., promoter, enhancer, coding region for a ribozyme binding site, 5′ of a coding sequence of a cellular protein. As defined above, the terms “control element” or “control sequence” refer collectively to promoter sequences, enhancers, coding regions for a ribozyme binding sites, upstream regulatory domains, origins of replication, nuclear localization sequences and the like. In some embodiments described below, the edits made to the cellular protein(s) in the present methods may also include—in addition to insertion of the peptide barcode/affinity tag/protease cleavage site—edits to 5′ or 3′ control elements (e.g., polyadenylation signals, transcription termination sequences and nuclear localization sequences).

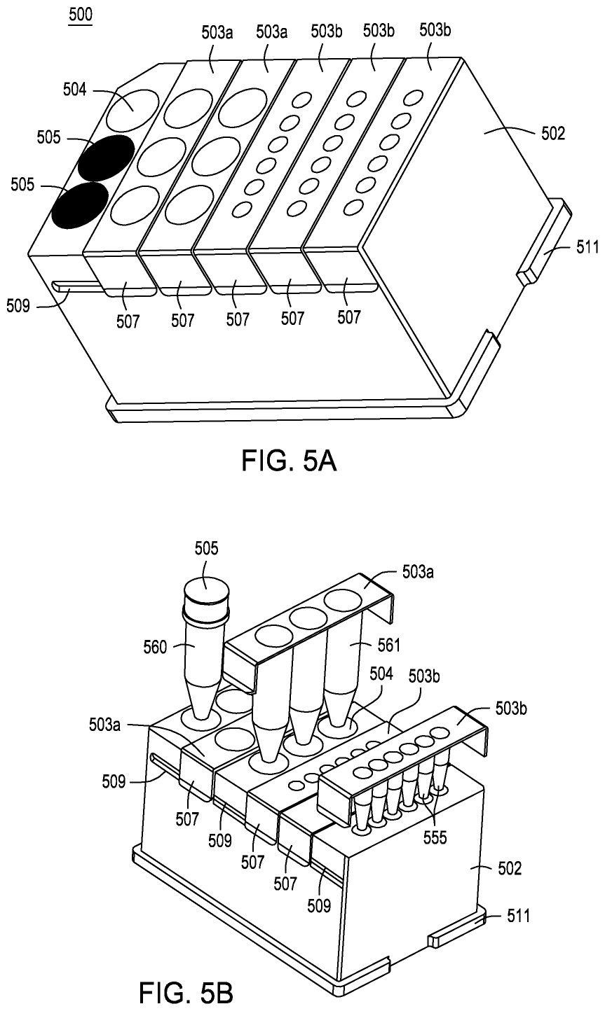

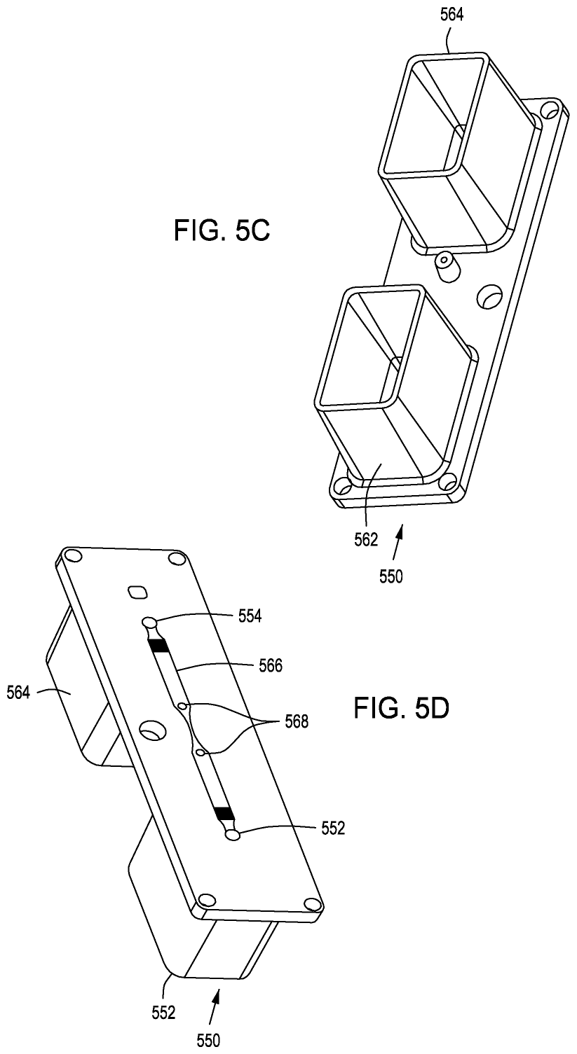

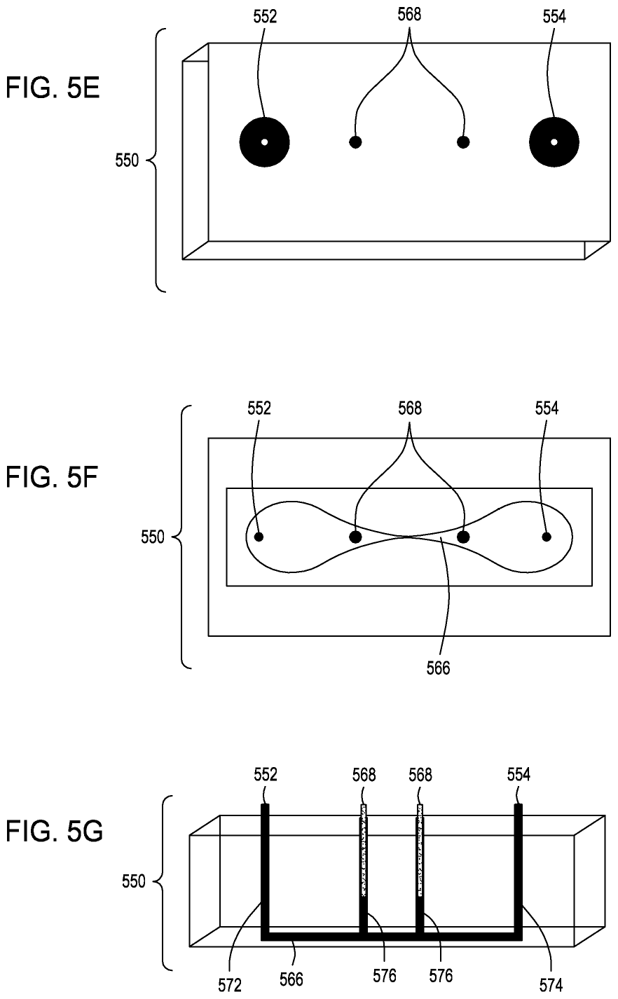

Element 124 of edited construct 120 is the coding sequence for the peptide barcode/affinity tag/protease cleavage site (or affinity tag/peptide barcode/protease cleavage site) that has been inserted 3′ the start codon “AUG” (not shown, but see element 142 of FIG. 1D) and 5′ to the rest of the coding sequence 126 of the cellular protein of interest. The order of the peptide barcode and affinity tag in element 124 does not matter; however, the protease cleavage site must be positioned 3′ to both the coding sequence for the peptide barcode and the coding sequence for the peptide affinity tag so that after protease cleavage, the peptide barcode remains associated with the affinity tag and the translated cellular protein is separated from the affinity tag and the peptide barcode.

The peptide barcodes are designed to be diverse (e.g., distinguishable from one another) in terms of first, mass-over-charge ratios so as to fall into the optimal m/z detection window of high-field orbitraps (550-850 m/z) and second, hydrophobicity to exploit the full separation capacity of a typical reverse-phase liquid chromotography system. The number of amino acids in the peptide barcodes ranges from three or four to twenty, or five to fifteen, or seven to twelve, depending both on the number of unique peptide barcodes needed for a library of editing cassettes (e.g., the size of the library) and to fit within the parameters needed for mass-over-charge ratio and hydrophobicity. In some aspects, the peptide barcodes comprise an arginine (R) residue as a sole positively charged residue, to support efficient ionization. Typically the peptide barcode does not contain cysteines or methionines to avoid oxidation and cross-linking, but may contain aspartate and glutamate to enhance solubility. Including proline in the peptide barcode facilitates collision-induced fragmentation. In one embodiment, the peptide barcodes encode the following amino acids: alanine (A), serine (S), threonine (T), asparagine (N), glutamine (Q), aspartic acid (D), glutamic acid (E), valine (V), leucine (L), phenylalanine (F), tyrosine (Y), tryptophan (W), glycine (G) and proline (P). In some instances, a list of designed peptide barcodes may be kept as a reference set with calculated mass and charge quantities and associated with the edit sequence or may be generated at the time of the edit sequence and associated therein. In some embodiments, more than one peptide barcode is associated with a specific edit but with distinct DNA identifying barcodes, allowing for redundant tracking of peptide barcodes and diversifying the barcode sequence to not inhibit the protein function. (See, e.g., Egloff, et al., doi.org/10.110/287813 (2018); and Miyamoto, et al., PLOS ONE, doi.org/10.1371/journal.pone.0215993 (2019).)

As described generally above, affinity tags are peptide sequences genetically “grafted” onto a protein—in the present instance, coding sequences for the affinity tags are edited into a coding sequence for a cellular protein along with the peptide barcode and protease cleavage site; that is, the coding sequence for an affinity tag is part of the barcode construct. In general, affinity tags are appended to proteins so that the tagged proteins can be purified from a crude biological extract using an affinity technique. Affinity tags include, e.g., polyhistidine tags, chitin binding protein, maltose binding protein, snap tags, flag tags, and glutathione-S-transferase as well as others known in the art.

The polyhistidine affinity tag, also known as a His-tag or His6, typically consists of six consecutive histidine residues but can vary in length from two to ten histidine residues. Polyhistidine is an ubiquitous affinity tag offered commercially by a number of companies and readily forms coordination bonds with immobilized transition metal ions (e.g., which facilitates immobilized metal affinity chromatography, “IMAC”), including Co²⁺, Cu²⁺, Ni²⁺, Zn²⁺, Ca²⁺ and Fe³⁺. Ni²⁺ is most commonly used, most often as a Ni(II)-nitrilotriacetic acid (Ni-NTA) (Qiagen, Hilden, Germany). Commercially-available IMAC resins typically are unaffected by protease or nuclease or nickase fusion activity and thus are appropriate for purification of proteins from crude cell lysates.

There are a number of other affinity tags known in the art suitable for use in the present compositions and methods. For example, Glutathoine S-Transferase (“GST”) is another affinity tag that can be used to purify proteins. GST fusion proteins can be purified by affinity chromatography on commercially-available glutathione (γ-glutamylcysteinylglycine) Sepharose. Maltose binding protein (“MBP”) is yet another affinity tag that may be used. MBP fusion proteins may be purified by affinity chromatography on commercially available cross-linked amylose resins. In yet another example, the calmodulin binding protein purification system utilizes a C-terminal fragment from muscle myosin light-chain kinase and is purified using a calmodulin affinity resin. The intein-chitin domain tag is a combination of a protein self-splicing element (intein) with a chitin-binding domain and allows for the purification of a tagged protein by affinity chromatography on chitin resin. Yet another affinity tag is the streptavidin/biotin-based tag which can be purified by affinity chromatography on avidin resin. FLAG-tags, or FLAG octapeptides are polypeptide protein tags that can be added to a protein. FLAG-tags are artificial antigens to which specific, high affinity monoclonal antibodies have been developed and can be used for protein purification by affinity chromatography. In addition to FLAG-tags, other affinity tags that can be purified with specific antibodies include alkaline phosphatase, AU1 epitope, AU5 epitope, bacteriophage T7 epitope, E2 epitope, human influenza hemagglutinin HSV epitope, KT3 epitope, Myc epitope, S1-tag, TrpE, and VSV-G.

As for the protease cleavage sites, one common protease is the Tobacco Etch Virus (“TEV”) protease. The TEV protease recognizes the cleavage sequence ENLYFQ\S, where “\′ denotes the cleaved peptide bond. Another common protease is thrombin, which recognizes the cleavage sequence LVPR\GS. One additional common protease of use is Factor Xa, which cleaves after the arginine residue in its preferred cleavage site IE\DGR.

FIG. 1C is a simplified diagram of a construct 130 for a coding sequence for a cellular protein after nucleic acid-guided editing takes place to add a peptide barcode /affinity tag/protease cleavage site (the “barcode construct”) to the 3′ end of the protein coding sequence. As in FIG. 1B, element 122 is a control element, e.g., promoter, enhancer, coding region for a ribozyme binding site, 5′ of a coding sequence for cellular protein 126. The coding sequence for the cellular protein 126 is followed by element 128—the barcode construct—which is 3′ to the stop codon (not seen) and comprises the protease cleavage site/peptide barcode/affinity tag. In the embodiment in FIG. 1C, the protease cleavage site is positioned 5′ to the peptide barcode and affinity tag so as to separate the peptide barcode and affinity tag from the cellular protein. In addition to inserting the protease cleavage site/peptide barcode/affinity (e.g., barcode construct), the edits made to the cellular protein(s) in the present methods may also include edits to 3′ control elements such as polyadenylation signals, transcription termination sequences and nuclear localization sequences.

FIG. 1D is an exemplary coding sequence 140 for a barcode construct to be inserted 3′ of the coding start codon and 5′ of the remainder of the coding sequence for the cellular protein. Element 142 is the AUG start codon for the cellular protein, element 144 is the coding sequence for a His6 tag, element 146 is the coding sequence for a 7-residue peptide barcode, and element 148 is the coding sequence for the TEV protease.

### Applications of Nucleic Acid-Guided Nuclease or Nickase Fusion Peptide Barcode Insertion Edits

As described above, using a set of short, curated peptide barcodes allows for linking protein expression patterns with edits made during nucleic acid-guided nuclease or nickase fusion editing. The barcode constructs—e.g., the constructs comprising the peptide barcode, the affinity tag and the protease cleavage site—are inserted via editing in the N-terminus or C-terminus of the coding regions of cellular proteins of interest using rationally-designed editing cassettes. The number of different editing cassettes (and thus different barcode constructs) in a library of editing cassettes can be as many as tens of thousands or more, providing opportunities for correlating genome edits with the level of protein translation, including edits to transcription and translation control sequences and/or edits to other regions of the genome; tracking changes in the level of protein translation subsequent to subjecting a population of cells to various conditions; as well as facilitating “one pot” cell-free synthesis pathway production and cell-free transcription-translation system production via bulk purification of proteins from millions of cells simultaneously.

In one application, the editing cassettes in addition to comprising an edit inserting a barcode construct into a region 5′ of a protein coding sequence additionally edits a ribosome binding site (“RBS”) 5′ of the protein coding sequence. The edit to the RBS may produce an RBS of alternative strength (e.g., lower or higher), where the effect on the translation of the targeted cellular protein can be quantified. The library of editing cassettes in this library may target a single cellular protein with different edits to the RBS—or promoter or enhancer elements or coding sequence of the cellular protein—or may target a several to hundreds of cellular proteins with two, three, four or more different edits to each protein. Again, each editing cassette comprising a barcode construct (and each peptide barcode in each barcode construct)) will code for a different peptide barcode so that the edit made may be correlated with the resulting translation level of cellular proteins.

In one embodiment of the methods herein, a technique referred to as isolation (or singulation), induction and normalization may be used. This technique is depicted in FIG. 6A and the description thereof. In this technique, cells are transformed, singulated, then allowed to grow for several to many doublings before editing is initiated using an inducible promoter driving transcription of the editing cassette. By singulating the cells, each cell is allowed to grow and form clonal colonies without having to compete against, e.g., untransformed cells; then, by allowing the transformed cells to form colonies before editing is initiated, the cells are able to form a “critical mass” in which at least some small number of cells (or more) are able to survive the double-stranded breaks that occur during editing. After editing is induced, all the colonies of cells are grown to “terminal size”—that is, all cell colonies are grown to roughly the same size—where there will be approximately the same number of cells from each colony no matter if the cells from a colony are edited cells, unedited cells, or cells where an edit impacts fitness (positively or negatively). The isolation (or singulation), induction and normalization technique is particularly useful in the methods described herein where the peptide barcodes are used to quantify cellular proteins. By negating the impact from unedited cells and fitness effects, the quantitation and relative level of expression of the cellular proteins is more reliable.

In yet another application, combination edits may be made. That is, instead of an editing plasmid or vector comprising a single editing cassette, two editing cassettes can be included in an editing vector where one editing cassette targets a cellular protein to insert the barcode construct 5′ or 3′ to a cellular protein of choice and the second editing cassette targets, e.g., other cellular proteins, transcription factors or control elements. Alternatively, cells of interest may be edited sequentially (e.g., recursively). Using combination editing, the effects of translation of the targeted cellular protein can be quantified after perturbation of virtually any location in the genome. For example, an edit may be made to a transcription factor in a cell, followed by (or simultaneously making) making tens, hundreds or thousands of edits inserting barcode constructs into cellular proteins of interest in a population of cells to assess the impact of the edit to the transcription factor on tens, hundreds or thousands of cellular proteins.

In some aspects of this application, the edit to the transcription factor is made in a cell population first where the cells are screened to be sure the desired edit has been made. This first-edited population of cells is then transformed with the editing cassettes comprising the barcode constructs to assess the impact of the edit to the transcription factor on tens, hundreds or thousands of cellular proteins. In using a sequential process, the edit to the transcription factor (or other cellular genome element) can be confirmed before making the edits to the cellular proteins. Additionally, the first-edited cells can be grown as a clonal population before making the second round of edits to be sure that the only variation in the cell population are the edits to the cellular proteins. In parallel, a population of cells without the edit to the transcription factor may be transformed with the tens, hundreds or thousands of barcode construct editing cassettes and the cellular proteins from each population can be quantified and compared.

In yet another application, tens, hundreds or thousands of barcode construct editing cassettes can be used to tag and purify cellular proteins in a particular metabolic or synthesis pathway, such as editing all proteins known in the glycolysis pathway, the citric acid cycle, translation factors, amino acyl-tRNA synthesis, and amino acid synthesis pathways. Editing cassettes are designed to insert barcode constructs 5′ or 3′ of each cellular protein in the pathway, which after editing and translation can be purified in bulk. In one example, transcription-translation systems may be created and purified in “one pot” in bulk. In 2001, Shimizu et al. (Nature Biotech., 19:751-55 (2001)) demonstrated that a defined, cell-free system called the “PURE” system (protein synthesis using recombinant elements) could be reconstituted from purified recombinant components. The PURE system has been used for genetic network engineering, recombinant DNA replication, molecular diagnostics and therapeutics. Recently, Lavickova and Maerkl (doi.org/10101/420570) demonstrated synthesis of a robust “one pot” PURE system by co-culturing all 36 tagged protein-producing E. coli clones in a single flask followed by isolating the transcription-translation proteins in a single Ni-NTA purification. In the present methods, a library of editing cassettes designed to insert barcode constructs into all proteins in a transcription-translation system is used to edit a population of cells. The affinity tag is then used to purify and “pull out” the edited proteins and the peptide barcode is used identify which proteins have been properly edited.

In another application, the barcode constructs could be inserted into proteins of interest for export from microbial cells where the barcode constructs are positioned between an export signal peptide and the coding sequence for the cellular protein. A signal peptide (sometimes referred to as signal sequence, targeting signal, localization signal, localization sequence, transit peptide, leader sequence or leader peptide) is a short peptide (usually 16-30 amino acids long) present at the N-terminus of a majority of newly-synthesized proteins that are destined toward the secretory pathway. These proteins include those that reside either inside certain organelles (e.g., the endoplasmic reticulum, Golgi or endosomes), secreted from the cell, or are inserted into most cellular membranes. Signal peptides function to prompt a cell to translocate the protein, usually to the cellular membrane. In prokaryotes, signal peptides direct the newly synthesized protein to the SecYEG protein-conducting channel, which is present in the plasma membrane. A homologous system exists in eukaryotes, where the signal peptide directs the newly synthesized protein to the Sec61 channel, which shares structural and sequence homology with SecYEG, but is present in the endoplasmic reticulum. The core of the signal peptide contains a long stretch of hydrophobic amino acids (about 5-16 residues long) that has a tendency to form a single alpha-helix.

In addition, the barcode constructs described herein are useful in two-hybrid systems. Two-hybrid screening (originally known as yeast two-hybrid system or Y2H) is a molecular biology technique used to discover protein-protein interactions and protein-DNA interactions by testing for physical interactions (such as binding) between two proteins or a single protein and a DNA molecule, respectively. The premise behind the test is the activation of downstream reporter gene(s) by the binding of a transcription factor onto an upstream activating sequence. For two-hybrid screening, the transcription factor is split into two separate fragments, called the DNA-binding domain (DBD or often also abbreviated as BD) and activating domain (AD). The BD is the domain responsible for binding to the upstream activating sequence and the AD is the domain responsible for the activation of transcription. The Y2H is thus a protein-fragment complementation assay. The key to the two-hybrid screen is that in most eukaryotic transcription factors, the activating and binding domains are modular and can function in proximity to each other without direct binding. This means that even though the transcription factor is split into two fragments, it can still activate transcription when the two fragments are indirectly connected. The most common screening approach is the yeast two-hybrid assay.

Similarly, phage display is a strategy for detecting protein-protein interaction and binding events by engineering, typically, filamentous phage M13 preferentially protein III or protein VIII or other coat proteins to display a protein fragment or protein of interest. Phage are produced in a pooled culture with variants in the binding domain or protein that are displayed on the phage, typically ran over a column displaying the other target of interest, for example an antibody/antigen pair, for which the phage is purified against the stationary phase. Importantly, the phage contains as part of its genome the gene encoding the protein fragment that is display, thus allowing a connection between the protein binding even and the underlying sequence of DNA encoding the fragment.

In both cases, protein interaction assays necessitate the tracking of the underlying sequence of DNA that encodes the protein. Applying the technology disclosed here, a protein can be encoded within the genome of interest, and placed under expression conditions to allow for that protein to be produced. Further, using genome editing technology allows for that protein to be mutated at 1, 2, 3, or more than 3 sites, with sequences from 1 mutation, 2, 3, or more than 3 to 60 nucleotides, or more than 60 nucleotides, to allow for insertion, substitutions, and deletions within a binding domain, such as the variable domain of an antibody, or nanobody, or other binding protein. Additionally, a second edit can simultaneously be introduced on the N-terminus or C-terminus of the protein encoded within the genome that is linked to the insertion/substitution/deletion of the variable binding domain, and thus allows for an association of an N-terminal or C-terminal His6 or other tag, a peptide barcode, and a protease cleavage site that all of the above described technologies would enable tracking and association of proteins to an underlying DNA sequence. Further then, production of this protein with the tag will be produced in cells and the cells will be lysed and the whole-cell lysate, or cleared lysate, or partially purified lysate will be subjected to flow over the stationary phase column with the other protein of interest attached. The protein captured by the column will be released and purified against a Nickel-NTA column or gel or similar, as known in the art, and the fragment containing the peptide barcode will be thus purified on the nickel column and eluted and subjected to mass spec analysis to determine the proteins that preferentially bound to the column. This peptide barcode can then be associated back to the genomic edit through the DNA barcode identifying the edit, and thus this method would allow rapid, high-throughput detection of the protein variant that binds to be sequenced and read out for subsequent analysis.

In yet another application, ribosomes have been engineered to generate orthogonal translation systems, having a modified anti-Shine-Delgarno (anti-SD) sequence that preferentially binds not the wild-type SD sequence but instead other engineered orthogonal messenger RNA (mRNA) sequences and thus translates according to the sequence of those mRNAs and not the native wild-type sequences. Further, these orthogonal translation ribosomes can be engineered in ways that are unlinked from cell viability. Thus, some ribosomes can be generated that have alternative 23S and 16S ribosomal RNA (rRNA) sequences, such as being modified to be linked by poly-A and poly-T linkers that tether the ribosomes. Further, the peptidyl transferase center may be modified in such ribosomes to enable alternative amino acid incorporation such as modified amino acids. However, limitations in the ribosome engineering have not allow for substantial screening to generate ribosomes that can incorporate these non-canonical amino acids efficiently. Using genome engineering, a set of ribosomes with modified peptidyl transferase centers, typically within nucleotide 1,500-2,600 of the 23S rRNA can be targeted for mutagenesis, or proteins near the catalytic core of the ribosome, or proteins near the exit tunnel or other ribosomal proteins can additionally modifying the translation apparatus. Analysis of such ribosome modifications can be assayed in cell-free systems by, for example, setting up an in vitro translation system with a tRNA that encodes for a non-canonical amino acid and is separately charged with that non-canonical amino acid through, for example, catalytic ribozymes [e.g. https://doi.org/10.1016/j.tibs.2014.07.005].

Separately, it has been shown that ribosomal protein L7/L12 is a late-stage assembly protein, and that can be used to purify ribosomes by His6-tagging the L7/L12 protein [https://www.ncbi.nlm.nih.gov/pmc/articles/PMC2632923/]. Once assembled, the L7/L12 protein remains stably bound to the ribosome and can be used as a link between purification of the protein and the behavior of the individual ribosome. Thus, using the described technology, 1, 2, 3, or more than 3 edits can be made to the ribosome, ribosomal proteins, or ribosomal RNA, and an additional edit can be made to introduce the His6-peptide barcode as described into the L7/L12 protein, with ribosomes purified, assayed for fidelity of protein production by, for example, producing a protein with a ribosomal pause sequence and assaying for or purifying only those ribosomes producing the protein with the modified amino acid to translate the protein containing that modified amino acid, and then cleavage and purification of the peptide barcode from L7/L12 that links the edits to that ribosome back to the sequences of the cell that were modified that produced that ribosome. Development of such ribosomes with efficient translation of non-canonical amino acids would then enable new technologies in therapeutic and industrial protein production that would be protected from degradation and immune response.

### Automated Cell Editing Instruments and Modules to Perform Nucleic Acid-Guided Nuclease or Nickase Fusion Editing in Cells

### Automated Cell Editing Instruments

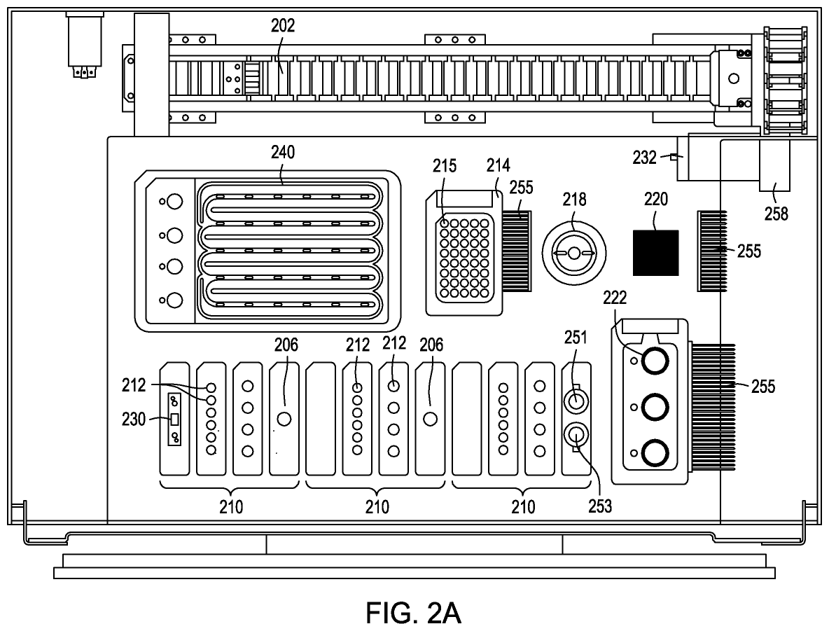

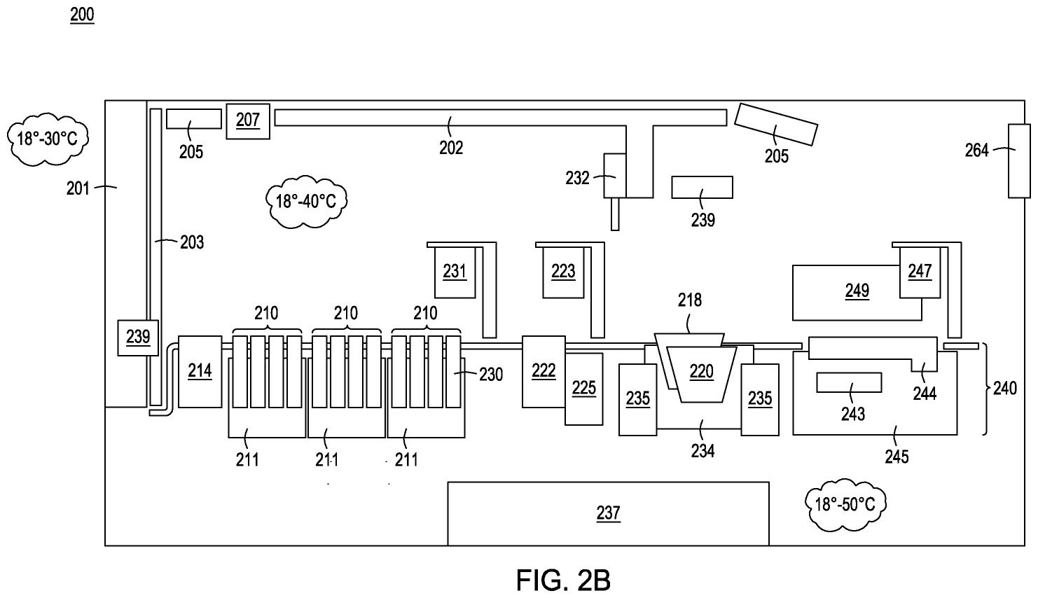

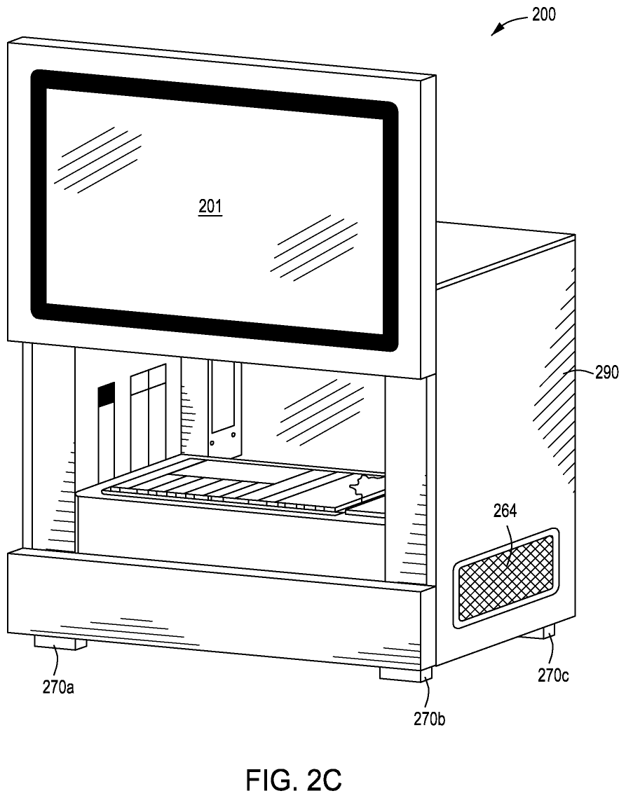

FIG. 2A depicts an exemplary automated multi-module cell processing instrument 200 to, e.g., perform targeted gene editing of live cells. The instrument 200, for example, may be and preferably is designed as a stand-alone desktop instrument for use within a laboratory environment. The instrument 200 may incorporate a mixture of reusable and disposable components for performing the various integrated processes in conducting automated genome cleavage and/or editing in cells without human intervention. Illustrated is a gantry 202, providing an automated mechanical motion system (actuator) (not shown) that supplies XYZ axis motion control to, e.g., an automated (i.e., robotic) liquid handling system 258 including, e.g., an air displacement pipettor 232 which allows for cell processing among multiple modules without human intervention. In some automated multi-module cell processing instruments, the air displacement pipettor 232 is moved by gantry 202 and the various modules and reagent cartridges remain stationary; however, in other embodiments, the liquid handling system 258 may stay stationary while the various modules and reagent cartridges are moved. Also included in the automated multi-module cell processing instrument 200 are reagent cartridges 210 comprising reservoirs 212 and transformation module 230 (e.g., a flow-through electroporation device as described in detail in relation to FIGS. 5B-5F), as well as wash reservoirs 206, cell input reservoir 251 and cell output reservoir 253. The wash reservoirs 206 may be configured to accommodate large tubes, for example, wash solutions, or solutions that are used often throughout an iterative process. Although two of the reagent cartridges 210 comprise a wash reservoir 206 in FIG. 2A, the wash reservoirs instead could be included in a wash cartridge where the reagent and wash cartridges are separate cartridges. In such a case, the reagent cartridge and wash cartridge may be identical except for the consumables (reagents or other components contained within the various inserts) inserted therein.

In some implementations, the reagent cartridges 210 are disposable kits comprising reagents and cells for use in the automated multi-module cell processing/editing instrument 200. For example, a user may open and position each of the reagent cartridges 210 comprising various desired inserts and reagents within the chassis of the automated multi-module cell editing instrument 200 prior to activating cell processing. Further, each of the reagent cartridges 210 may be inserted into receptacles in the chassis having different temperature zones appropriate for the reagents contained therein.

Also illustrated in FIG. 2A is the robotic liquid handling system 258 including the gantry 202 and air displacement pipettor 232. In some examples, the robotic handling system 258 may include an automated liquid handling system such as those manufactured by Tecan Group Ltd. of Mannedorf, Switzerland, Hamilton Company of Reno, Nev. (see, e.g., WO2018015544A1), or Beckman Coulter, Inc. of Fort Collins, Colo. (see, e.g., US20160018427A1). Pipette tips may be provided in a pipette transfer tip supply (not shown) for use with the air displacement pipettor 232. The robotic liquid handling system allows for the transfer of liquids between modules without human intervention.

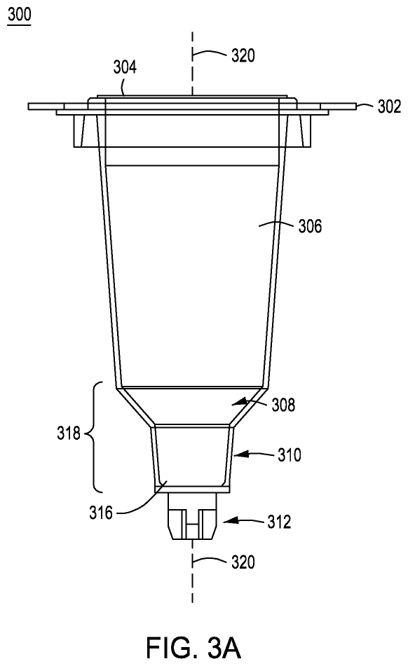

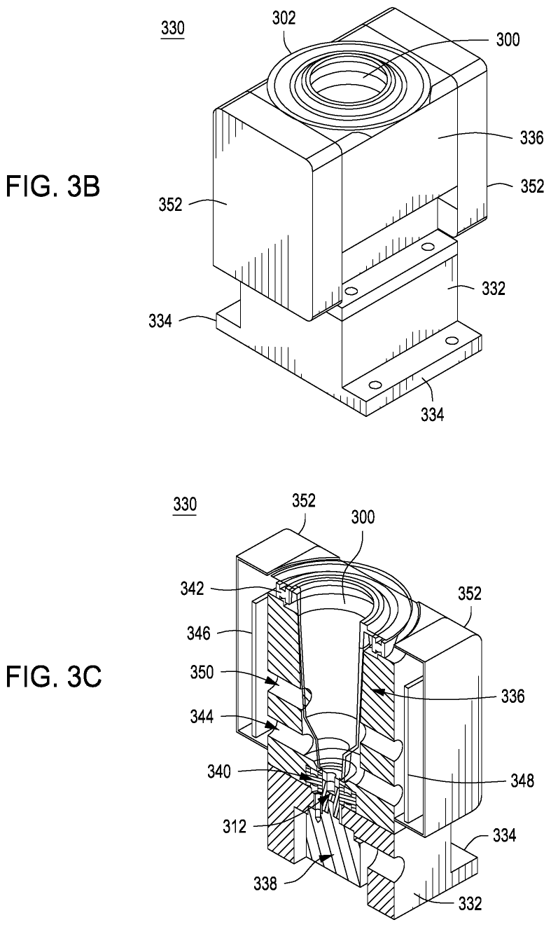

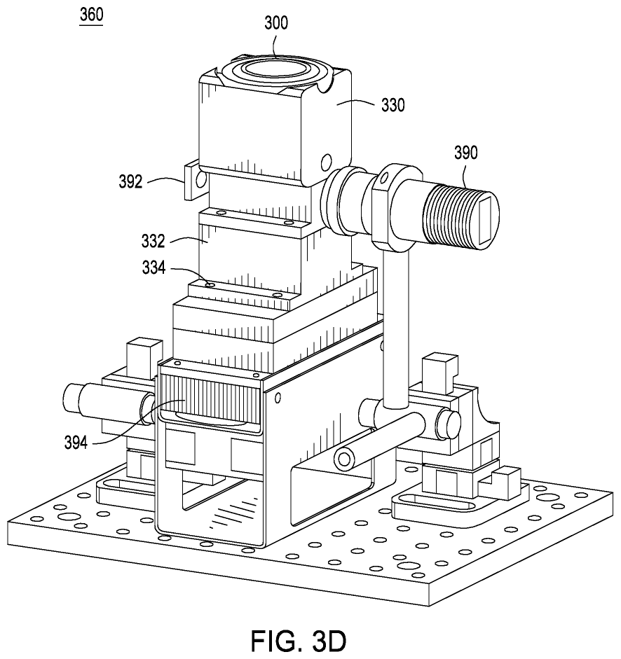

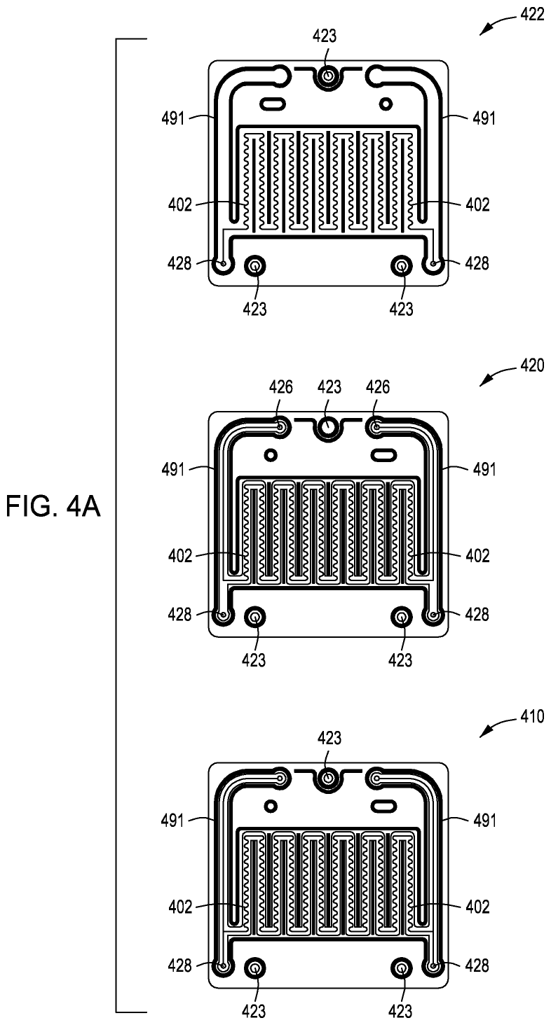

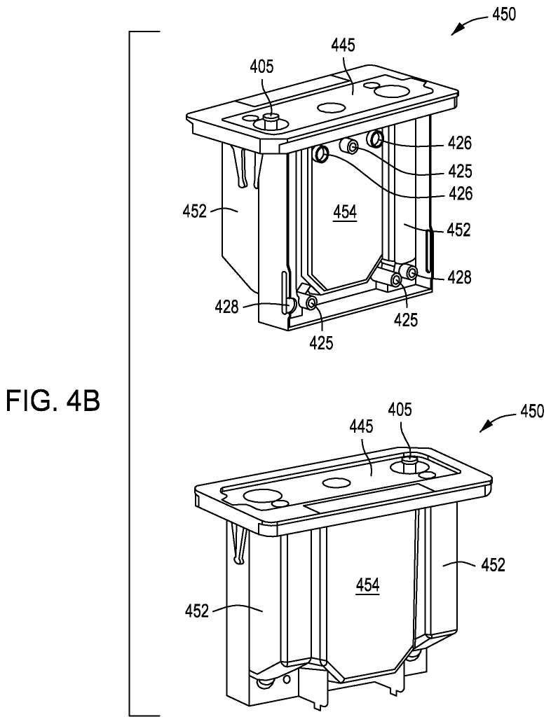

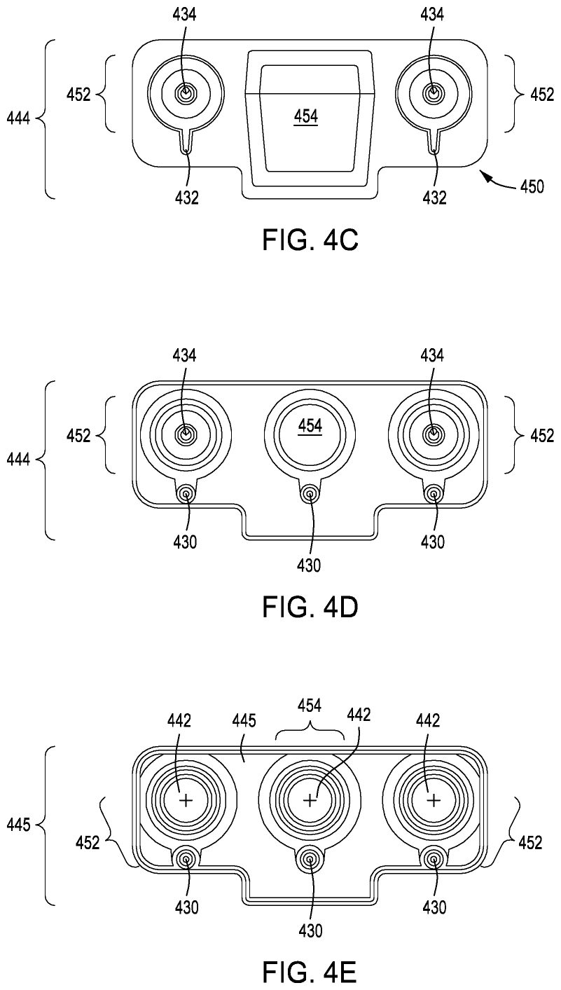

Inserts or components of the reagent cartridges 210, in some implementations, are marked with machine-readable indicia (not shown), such as bar codes, for recognition by the robotic handling system 258. For example, the robotic liquid handling system 258 may scan one or more inserts within each of the reagent cartridges 210 to confirm contents. In other implementations, machine-readable indicia may be marked upon each reagent cartridge 210, and a processing system (not shown, but see element 237 of FIG. 2B) of the automated multi-module cell editing instrument 200 may identify a stored materials map based upon the machine-readable indicia. In the embodiment illustrated in FIG. 2A, a cell growth module comprises a cell growth vial 218 (described in greater detail below in relation to FIGS. 3A-3D). Additionally seen is the TFF module 222 (described above in detail in relation to FIGS. 4A-4E). Also illustrated as part of the automated multi-module cell processing instrument 200 of FIG. 2A is a singulation module 240 (e.g., a solid wall isolation, incubation and normalization device (SWIIN device) is shown here) described herein in relation to FIGS. 6C-6F, served by, e.g., robotic liquid handing system 258 and air displacement pipettor 232. Additionally seen is a selection module 220. Also note the placement of three heatsinks 255.

FIG. 2B is a simplified representation of the contents of the exemplary multi-module cell processing instrument 200 depicted in FIG. 2A. Cartridge-based source materials (such as in reagent cartridges 210), for example, may be positioned in designated areas on a deck of the instrument 200 for access by an air displacement pipettor 232. The deck of the multi-module cell processing instrument 200 may include a protection sink such that contaminants spilling, dripping, or overflowing from any of the modules of the instrument 200 are contained within a lip of the protection sink. Also seen are reagent cartridges 210, which are shown disposed with thermal assemblies 211 which can create temperature zones appropriate for different regions. Note that one of the reagent cartridges also comprises a flow-through electroporation device 230 (FTEP), served by FTEP interface (e.g., manifold arm) and actuator 231. Also seen is TFF module 222 with adjacent thermal assembly 225, where the TFF module is served by TFF interface (e.g., manifold arm) and actuator 223. Thermal assemblies 225, 235, and 245 encompass thermal electric devices such as Peltier devices, as well as heatsinks, fans and coolers. The rotating growth vial 218 is within a growth module 234, where the growth module is served by two thermal assemblies 235. Selection module is seen at 220. Also seen is the SWIIN module 240, comprising a SWIIN cartridge 244, where the SWIIN module also comprises a thermal assembly 245, illumination 243 (in this embodiment, backlighting), evaporation and condensation control 249, and where the SWIIN module is served by SWIIN interface (e.g., manifold arm) and actuator 247. Also seen in this view is touch screen display 201, display actuator 203, illumination 205 (one on either side of multi-module cell processing instrument 200), and cameras 239 (one illumination device on either side of multi-module cell processing instrument 200). Finally, element 237 comprises electronics, such as circuit control boards, high-voltage amplifiers, power supplies, and power entry; as well as pneumatics, such as pumps, valves and sensors.

FIG. 2C illustrates a front perspective view of multi-module cell processing instrument 200 for use in as a desktop version of the automated multi-module cell editing instrument 200. For example, a chassis 290 may have a width of about 24-48 inches, a height of about 24-48 inches and a depth of about 24-48 inches. Chassis 290 may be and preferably is designed to hold all modules and disposable supplies used in automated cell processing and to perform all processes required without human intervention; that is, chassis 290 is configured to provide an integrated, stand-alone automated multi-module cell processing instrument. As illustrated in FIG. 2C, chassis 290 includes touch screen display 201, cooling grate 264, which allows for air flow via an internal fan (not shown). The touch screen display provides information to a user regarding the processing status of the automated multi-module cell editing instrument 200 and accepts inputs from the user for conducting the cell processing. In this embodiment, the chassis 290 is lifted by adjustable feet 270a, 270b, 270c and 270d (feet 270a-270c are shown in this FIG. 2C). Adjustable feet 270a-270d, for example, allow for additional air flow beneath the chassis 290.

Inside the chassis 290, in some implementations, will be most or all of the components described in relation to FIGS. 2A and 2B, including the robotic liquid handling system disposed along a gantry, reagent cartridges 210 including a flow-through electroporation device, a rotating growth vial 218 in a cell growth module 234, a tangential flow filtration module 222, a SWIIN module 240 as well as interfaces and actuators for the various modules. In addition, chassis 290 houses control circuitry, liquid handling tubes, air pump controls, valves, sensors, thermal assemblies (e.g., heating and cooling units) and other control mechanisms. For examples of multi-module cell editing instruments, see U.S. Pat. No. 10,253,316, issued 9 Apr. 2019; U.S. Pat. No. 10,329,559, issued 25 Jun. 2019; U.S. Pat. No. 10,323,242, issued 18 Jun. 2019; U.S. Pat. No. 10,421,959, issued 24 Sep. 2019; U.S. Pat. No. 10,465,185, issued 5 Nov. 2019; U.S. Pat. No. 10,519,437, issued 31 Dec. 2019; U.S. Pat. No. 10,584,333, issued 10 Mar. 2020; U.S. Pat. No. 10,584,334, issued 10 Mar. 2020; U.S. Pat. No. 10,647,982, issued 12 May 2020; and U.S. Pat. No. 10,689,645, issued 23 Jun. 2020, all of which are herein incorporated by reference in their entirety.

### The Rotating Cell Growth Module

FIG. 3A shows one embodiment of a rotating growth vial 300 for use with the cell growth device and in the automated multi-module cell processing instruments described herein for growing cells (e.g., bacterial, yeast or animal) in suspension. Bacterial and yeast cells are typically grown in suspension. Growing mammalian cells in suspension (e.g., even adherent cells) can be effected in various forms. Adherent cells that typically are grown in 2D cultures when grown in suspension often aggregate into “clumps.” For example, some mammalian cells grow well as aggregates in suspension, and are most healthy growing in aggregates of 50-300 microns in size, starting off as smaller aggregates 30-50 microns in size. Mammalian cells are typically grown in culture 3-5 days between passaging and the larger aggregates are broken into smaller aggregates by filtering them, e.g., through a cell strainer (e.g., a sieve) with a 37 micron filter. The mammalian cells can grow indefinitely in 3D aggregates as long as they are passaged into smaller aggregates when the aggregates become 300-400 microns in size.

An alternative to growing cells in 3D aggregates is growing cells on microcarriers. Generally, microcarriers are nonporous (comprised of pore sizes range from 0-20 nm), microporous (comprised of pore sizes range from 20 nm-1 micron), and macroporous (comprised of pore sizes range from 1-50 microns) microcarriers comprising natural organic materials such as, e.g., gelatin, collagen, alginate, agarose, chitosan, and cellulose, synthetic polymeric materials such as, e.g., polystyrene, polyacrylates such as polyacrylamide, polyamidoamine (PAMAM), polyethylene oxide (PEO/PEG), poly(N-isopropylacrylamide) (PNIPAM), polycaprolactone (PCL), polylactic acid (PLA), and polyglycolic acid (PGA), inorganic materials such as, e.g., silica, silicon, mica, quartz and silicone, as well as mixtures of natural, polymeric materials, cross-linked polymeric materials, and inorganic materials etc. on which animal cells can grow. Microcarriers useful for the methods herein typically range in size from 30-1200 microns in diameter and more typically range in size from 40-200 or from 50-150 microns in diameter.

Finally, another option for growing mammalian cells for editing in the compositions, methods, modules and automated instruments described herein is growing single cells in suspension using a specialized medium such as that developed by Accellta™ (Haifa, Israel). Cells grown in this medium must be adapted to this process over many cell passages; however, once adapted the cells can be grown to a density of >40 million cells/ml and expanded 50-100× in approximately a week, depending on cell type.

The rotating growth vial 300 is an optically-transparent container having an open end 304 for receiving liquid media and cells, a central vial region 306 that defines the primary container for growing cells, a tapered-to-constricted region 318 defining at least one light path 310, a closed end 316, and a drive engagement mechanism 312. The rotating growth vial 300 has a central longitudinal axis 320 around which the vial rotates, and the light path 310 is generally perpendicular to the longitudinal axis of the vial. The first light path 310 is positioned in the lower constricted portion of the tapered-to-constricted region 318. Optionally, some embodiments of the rotating growth vial 300 have a second light path 308 in the tapered region of the tapered-to-constricted region 318. Both light paths in this embodiment are positioned in a region of the rotating growth vial that is constantly filled with the cell culture (cells+growth media) and are not affected by the rotational speed of the growth vial. The first light path 310 is shorter than the second light path 308 allowing for sensitive measurement of OD values when the OD values of the cell culture in the vial are at a high level (e.g., later in the cell growth process), whereas the second light path 308 allows for sensitive measurement of OD values when the OD values of the cell culture in the vial are at a lower level (e.g., earlier in the cell growth process).

The drive engagement mechanism 312 engages with a motor (not shown) to rotate the vial. In some embodiments, the motor drives the drive engagement mechanism 312 such that the rotating growth vial 300 is rotated in one direction only, and in other embodiments, the rotating growth vial 300 is rotated in a first direction for a first amount of time or periodicity, rotated in a second direction (i.e., the opposite direction) for a second amount of time or periodicity, and this process may be repeated so that the rotating growth vial 300 (and the cell culture contents) are subjected to an oscillating motion. Further, the choice of whether the culture is subjected to oscillation and the periodicity therefor may be selected by the user. The first amount of time and the second amount of time may be the same or may be different. The amount of time may be 1, 2, 3, 4, 5, or more seconds, or may be 1, 2, 3, 4 or more minutes. In another embodiment, in an early stage of cell growth the rotating growth vial 400 may be oscillated at a first periodicity (e.g., every 60 seconds), and then a later stage of cell growth the rotating growth vial 300 may be oscillated at a second periodicity (e.g., every one second) different from the first periodicity.

The rotating growth vial 300 may be reusable or, preferably, the rotating growth vial is consumable. In some embodiments, the rotating growth vial is consumable and is presented to the user pre-filled with growth medium, where the vial is hermetically sealed at the open end 304 with a foil seal. A medium-filled rotating growth vial packaged in such a manner may be part of a kit for use with a stand-alone cell growth device or with a cell growth module that is part of an automated multi-module cell processing system. To introduce cells into the vial, a user need only pipette up a desired volume of cells and use the pipette tip to punch through the foil seal of the vial. Open end 304 may optionally include an extended lip 302 to overlap and engage with the cell growth device. In automated systems, the rotating growth vial 300 may be tagged with a barcode or other identifying means that can be read by a scanner or camera (not shown) that is part of the automated system.

The volume of the rotating growth vial 300 and the volume of the cell culture (including growth medium) may vary greatly, but the volume of the rotating growth vial 300 must be large enough to generate a specified total number of cells. In practice, the volume of the rotating growth vial 300 may range from 1-250 mL, 2-100 mL, from 5-80 mL, 10-50 mL, or from 12-35 mL. Likewise, the volume of the cell culture (cells+growth media) should be appropriate to allow proper aeration and mixing in the rotating growth vial 300. Proper aeration promotes uniform cellular respiration within the growth media. Thus, the volume of the cell culture should be approximately 5-85% of the volume of the growth vial or from 20-60% of the volume of the growth vial. For example, for a 30 mL growth vial, the volume of the cell culture would be from about 1.5 mL to about 26 mL, or from 6 mL to about 18 mL.

The rotating growth vial 300 preferably is fabricated from a bio-compatible optically transparent material—or at least the portion of the vial comprising the light path(s) is transparent. Additionally, material from which the rotating growth vial is fabricated should be able to be cooled to about 4° C. or lower and heated to about 55° C. or higher to accommodate both temperature-based cell assays and long-term storage at low temperatures. Further, the material that is used to fabricate the vial must be able to withstand temperatures up to 55° C. without deformation while spinning. Suitable materials include cyclic olefin copolymer (COC), glass, polyvinyl chloride, polyethylene, polyamide, polypropylene, polycarbonate, poly(methyl methacrylate (PMMA), polysulfone, polyurethane, and co-polymers of these and other polymers. Preferred materials include polypropylene, polycarbonate, or polystyrene. In some embodiments, the rotating growth vial is inexpensively fabricated by, e.g., injection molding or extrusion.

FIG. 3B is a perspective view of one embodiment of a cell growth device 330. FIG. 3C depicts a cut-away view of the cell growth device 330 from FIG. 3B. In both figures, the rotating growth vial 300 is seen positioned inside a main housing 336 with the extended lip 302 of the rotating growth vial 300 extending above the main housing 336. Additionally, end housings 352, a lower housing 332 and flanges 334 are indicated in both figures. Flanges 334 are used to attach the cell growth device 330 to heating/cooling means or other structure (not shown). FIG. 3C depicts additional detail. In FIG. 3C, upper bearing 342 and lower bearing 340 are shown positioned within main housing 336. Upper bearing 342 and lower bearing 340 support the vertical load of rotating growth vial 300. Lower housing 332 contains the drive motor 338. The cell growth device 330 of FIG. 3C comprises two light paths: a primary light path 344, and a secondary light path 350. Light path 344 corresponds to light path 310 positioned in the constricted portion of the tapered-to-constricted portion of the rotating growth vial 300, and light path 350 corresponds to light path 308 in the tapered portion of the tapered-to-constricted portion of the rotating growth vial 300. Light paths 310 and 308 are not shown in FIG. 3C but may be seen in FIG. 3A. In addition to light paths 344 and 340, there is an emission board 348 to illuminate the light path(s), and detector board 346 to detect the light after the light travels through the cell culture liquid in the rotating growth vial 300.

The motor 338 engages with drive mechanism 312 and is used to rotate the rotating growth vial 300. In some embodiments, motor 338 is a brushless DC type drive motor with built-in drive controls that can be set to hold a constant revolution per minute (RPM) between 0 and about 3000 RPM. Alternatively, other motor types such as a stepper, servo, brushed DC, and the like can be used. Optionally, the motor 338 may also have direction control to allow reversing of the rotational direction, and a tachometer to sense and report actual RPM. The motor is controlled by a processor (not shown) according to, e.g., standard protocols programmed into the processor and/or user input, and the motor may be configured to vary RPM to cause axial precession of the cell culture thereby enhancing mixing, e.g., to prevent cell aggregation, increase aeration, and optimize cellular respiration.

Main housing 336, end housings 352 and lower housing 332 of the cell growth device 330 may be fabricated from any suitable, robust material including aluminum, stainless steel, and other thermally conductive materials, including plastics. These structures or portions thereof can be created through various techniques, e.g., metal fabrication, injection molding, creation of structural layers that are fused, etc. Whereas the rotating growth vial 300 is envisioned in some embodiments to be reusable, but preferably is consumable, the other components of the cell growth device 330 are preferably reusable and function as a stand-alone benchtop device or as a module in a multi-module cell processing system.

The processor (not shown) of the cell growth device 330 may be programmed with information to be used as a “blank” or control for the growing cell culture. A “blank” or control is a vessel containing cell growth medium only, which yields 100% transmittance and 0 OD, while the cell sample will deflect light rays and will have a lower percent transmittance and higher OD. As the cells grow in the media and become denser, transmittance will decrease and OD will increase. The processor (not shown) of the cell growth device 330—may be programmed to use wavelength values for blanks commensurate with the growth media typically used in cell culture (whether, e.g., mammalian cells, bacterial cells, animal cells, yeast cells, etc.). Alternatively, a second spectrophotometer and vessel may be included in the cell growth device 330, where the second spectrophotometer is used to read a blank at designated intervals.

FIG. 3D illustrates a cell growth assembly 360 as part of an assembly comprising the cell growth device 330 of FIG. 3B coupled to light source 390, detector 392, and thermal components 394. The rotating growth vial 300 is inserted into the cell growth device. Components of the light source 390 and detector 392 (e.g., such as a photodiode with gain control to cover 5-log) are coupled to the main housing of the cell growth device. The lower housing 332 that houses the motor that rotates the rotating growth vial 300 is illustrated, as is one of the flanges 334 that secures the cell growth device 330 to the assembly. Also, the thermal components 394 illustrated are a Peltier device or thermoelectric cooler. In this embodiment, thermal control is accomplished by attachment and electrical integration of the cell growth device 330 to the thermal components 394 via the flange 334 on the base of the lower housing 332. Thermoelectric coolers are capable of “pumping” heat to either side of a junction, either cooling a surface or heating a surface depending on the direction of current flow. In one embodiment, a thermistor is used to measure the temperature of the main housing and then, through a standard electronic proportional-integral-derivative (PID) controller loop, the rotating growth vial 300 is controlled to approximately +/−0.5° C.

In use, cells are inoculated (cells can be pipetted, e.g., from an automated liquid handling system or by a user) into pre-filled growth media of a rotating growth vial 300 by piercing though the foil seal or film. The programmed software of the cell growth device 330 sets the control temperature for growth, typically 30° C., then slowly starts the rotation of the rotating growth vial 300. The cell/growth media mixture slowly moves vertically up the wall due to centrifugal force allowing the rotating growth vial 300 to expose a large surface area of the mixture to a normal oxygen environment. The growth monitoring system takes either continuous readings of the OD or OD measurements at pre-set or pre-programmed time intervals. These measurements are stored in internal memory and if requested the software plots the measurements versus time to display a growth curve. If enhanced mixing is required, e.g., to optimize growth conditions, the speed of the vial rotation can be varied to cause an axial precession of the liquid, and/or a complete directional change can be performed at programmed intervals. The growth monitoring can be programmed to automatically terminate the growth stage at a pre-determined OD, and then quickly cool the mixture to a lower temperature to inhibit further growth.

One application for the cell growth device 330 is to constantly measure the optical density of a growing cell culture. One advantage of the described cell growth device is that optical density can be measured continuously (kinetic monitoring) or at specific time intervals; e.g., every 5, 10, 15, 20, 30 45, or 60 seconds, or every 1, 2, 3, 4, 5, 6, 7, 8, 9, or 10 minutes. While the cell growth device 330 has been described in the context of measuring the optical density (OD) of a growing cell culture, it should, however, be understood by a skilled artisan given the teachings of the present specification that other cell growth parameters can be measured in addition to or instead of cell culture OD. As with optional measure of cell growth in relation to the solid wall device or module described supra, spectroscopy using visible, UV, or near infrared (NIR) light allows monitoring the concentration of nutrients and/or wastes in the cell culture and other spectroscopic measurements may be made; that is, other spectral properties can be measured via, e.g., dielectric impedance spectroscopy, visible fluorescence, fluorescence polarization, or luminescence. Additionally, the cell growth device 430 may include additional sensors for measuring, e.g., dissolved oxygen, carbon dioxide, pH, conductivity, and the like. For additional details regarding rotating growth vials and cell growth devices see U.S. Pat. No. 10,435,662, issued 8 Oct. 2019; U.S. Pat. No. 10,443,031, issued 15 Oct. 2019; U.S. Pat. No. 10,590,375, issued 17 Mar. 2020; and U.S. Ser. No. 16/780,640, filed 3 Feb. 2020; and Ser. No. 16/836,664, filed 31 Mar. 2020.

### The Cell Concentration Module

As described above in relation to the rotating growth vial and cell growth module, in order to obtain an adequate number of cells for transformation or transfection, cells typically are grown to a specific optical density in medium appropriate for the growth of the cells of interest; however, for effective transformation or transfection, it is desirable to decrease the volume of the cells as well as render the cells competent via buffer or medium exchange. Thus, one sub-component or module that is desired in automated, integrated, multi-module instruments for the processes listed above is a module or component that can grow, perform buffer exchange, and/or concentrate cells and render them competent so that they may be transformed or transfected with the nucleic acids needed for engineering or editing the cell's genome.

FIG. 4A shows a retentate member 422 (top), permeate member 420 (middle) and a tangential flow assembly 410 (bottom) comprising the retentate member 422, membrane 424 (not seen in FIG. 4A), and permeate member 420 (also not seen). In FIG. 4A, retentate member 422 comprises a tangential flow channel 402, which has a serpentine configuration that initiates at one lower corner of retentate member 422—specifically at retentate port 428—traverses across and up then down and across retentate member 422, ending in the other lower corner of retentate member 422 at a second retentate port 428. Also seen on retentate member 422 are energy directors 491, which circumscribe the region where a membrane or filter (not seen in this FIG. 4A) is seated, as well as interdigitate between areas of channel 402. Energy directors 491 in this embodiment mate with and serve to facilitate ultrasonic welding or bonding of retentate member 422 with permeate/filtrate member 420 via the energy director component 491 on permeate/filtrate member 420 (at right). Additionally, countersinks 423 can be seen, two on the bottom one at the top middle of retentate member 422. Countersinks 423 are used to couple a tangential flow assembly to a reservoir assembly (not seen in this FIG. 4A but see FIG. 4B).

Permeate/filtrate member 420 is seen in the middle of FIG. 4A and comprises, in addition to energy director 491, through-holes for retentate ports 428 at each bottom corner (which mate with the through-holes for retentate ports 428 at the bottom corners of retentate member 422), as well as a tangential flow channel 402 and two permeate/filtrate ports 426 positioned at the top and center of permeate member 420. The tangential flow channel 402 structure in this embodiment has a serpentine configuration and an undulating geometry, although other geometries may be used. Permeate member 420 also comprises countersinks 423, coincident with the countersinks 423 on retentate member 420.

On the bottom of FIG. 4A is a tangential flow assembly 410 comprising the retentate member 422 and permeate member 420 seen in this FIG. 4A. In this view, retentate member 422 is “on top” of the view, a membrane (not seen in this view of the assembly) would be adjacent and under retentate member 422 and permeate member 420 (also not seen in this view of the assembly) is adjacent to and beneath the membrane. Again countersinks 423 are seen, where the countersinks in the retentate member 422 and the permeate member 420 are coincident and configured to mate with threads or mating elements for the countersinks disposed on a reservoir assembly (not seen in FIG. 4A but see FIG. 4B).

A membrane or filter is disposed between the retentate and permeate members, where fluids can flow through the membrane but cells cannot and are thus retained in the flow channel disposed in the retentate member. Filters or membranes appropriate for use in the TFF device/module are those that are solvent resistant, are contamination free during filtration, and are able to retain the types and sizes of cells of interest. For example, in order to retain small cell types such as bacterial cells, pore sizes can be as low as 0.2 μm, however for other cell types, the pore sizes can be as high as 20 μm. Indeed, the pore sizes useful in the TFF device/module include filters with sizes from 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.36 μm, 0.37 μm, 0.38 μm, 0.39 μm, 0.40 μm, 0.41 μm, 0.42 μm, 0.43 μm, 0.44 μm, 0.45 μm, 0.46 μm, 0.47 μm, 0.48 μm, 0.49 μm, 0.50 μm and larger. The filters may be fabricated from any suitable non-reactive material including cellulose mixed ester (cellulose nitrate and acetate) (CME), polycarbonate (PC), polyvinylidene fluoride (PVDF), polyethersulfone (PES), polytetrafluoroethylene (PTFE), nylon, glass fiber, or metal substrates as in the case of laser or electrochemical etching.

The length of the channel structure 402 may vary depending on the volume of the cell culture to be grown and the optical density of the cell culture to be concentrated. The length of the channel structure typically is from 60 mm to 300 mm, or from 70 mm to 200 mm, or from 80 mm to 100 mm. The cross-section configuration of the flow channel 402 may be round, elliptical, oval, square, rectangular, trapezoidal, or irregular. If square, rectangular, or another shape with generally straight sides, the cross section may be from about 10 μm to 1000 μm wide, or from 200 μm to 800 μm wide, or from 300 μm to 700 μm wide, or from 400 μm to 600 μm wide; and from about 10 μm to 1000 μm high, or from 200 μm to 800 μm high, or from 300 μm to 700 μm high, or from 400 μm to 600 μm high. If the cross section of the flow channel 402 is generally round, oval or elliptical, the radius of the channel may be from about 50 μm to 1000 μm in hydraulic radius, or from 5 μm to 800 μm in hydraulic radius, or from 200 μm to 700 μm in hydraulic radius, or from 300 μm to 600 μm wide in hydraulic radius, or from about 200 to 500 μm in hydraulic radius. Moreover, the volume of the channel in the retentate 422 and permeate 420 members may be different depending on the depth of the channel in each member.

FIG. 4B shows front perspective (right) and rear perspective (left) views of a reservoir assembly 450 configured to be used with the tangential flow assembly 410 seen in FIG. 4A. Seen in the front perspective view (e.g., “front” being the side of reservoir assembly 450 that is coupled to the tangential flow assembly 410 seen in FIG. 4A) are retentate reservoirs 452 on either side of permeate reservoir 454. Also seen are permeate ports 426, retentate ports 428, and three threads or mating elements 425 for countersinks 423 (countersinks 423 not seen in this FIG. 4B). Threads or mating elements 425 for countersinks 423 are configured to mate or couple the tangential flow assembly 410 (seen in FIG. 4A) to reservoir assembly 450. Alternatively or in addition, fasteners, sonic welding or heat stakes may be used to mate or couple the tangential flow assembly 410 to reservoir assembly 450. In addition is seen gasket 445 covering the top of reservoir assembly 450. Gasket 445 is described in detail in relation to FIG. 4E. At left in FIG. 4B is a rear perspective view of reservoir assembly 1250, where “rear” is the side of reservoir assembly 450 that is not coupled to the tangential flow assembly. Seen are retentate reservoirs 452, permeate reservoir 454, and gasket 445.

The TFF device may be fabricated from any robust material in which channels (and channel branches) may be milled including stainless steel, silicon, glass, aluminum, or plastics including cyclic-olefin copolymer (COC), cyclo-olefin polymer (COP), polystyrene, polyvinyl chloride, polyethylene, polyamide, polyethylene, polypropylene, acrylonitrile butadiene, polycarbonate, polyetheretheketone (PEEK), poly(methyl methylacrylate) (PMMA), polysulfone, and polyurethane, and co-polymers of these and other polymers. If the TFF device/module is disposable, preferably it is made of plastic. In some embodiments, the material used to fabricate the TFF device/module is thermally-conductive so that the cell culture may be heated or cooled to a desired temperature. In certain embodiments, the TFF device is formed by precision mechanical machining, laser machining, electro discharge machining (for metal devices); wet or dry etching (for silicon devices); dry or wet etching, powder or sandblasting, photostructuring (for glass devices); or thermoforming, injection molding, hot embossing, or laser machining (for plastic devices) using the materials mentioned above that are amenable to this mass production techniques.

FIG. 4C depicts a top-down view of the reservoir assemblies 450 shown in FIG. 4B. FIG. 4D depicts a cover 444 for reservoir assembly 450 shown in FIG. 4B and 4E depicts a gasket 445 that in operation is disposed on cover 444 of reservoir assemblies 450 shown in FIG. 4B. FIG. 4C is a top-down view of reservoir assembly 450, showing the tops of the two retentate reservoirs 452, one on either side of permeate reservoir 454. Also seen are grooves 432 that will mate with a pneumatic port (not shown), and fluid channels 434 that reside at the bottom of retentate reservoirs 452, which fluidically couple the retentate reservoirs 452 with the retentate ports 428 (not shown), via the through-holes for the retentate ports in permeate member 420 and membrane 424 (also not shown). FIG. 4D depicts a cover 444 that is configured to be disposed upon the top of reservoir assembly 450. Cover 444 has round cut-outs at the top of retentate reservoirs 452 and permeate/filtrate reservoir 454. Again at the bottom of retentate reservoirs 452 fluid channels 434 can be seen, where fluid channels 434 fluidically couple retentate reservoirs 452 with the retentate ports 428 (not shown). Also shown are three pneumatic ports 430 for each retentate reservoir 452 and permeate/filtrate reservoir 454. FIG. 4E depicts a gasket 445 that is configures to be disposed upon the cover 444 of reservoir assembly 450. Seen are three fluid transfer ports 442 for each retentate reservoir 452 and for permeate/filtrate reservoir 454. Again, three pneumatic ports 430, for each retentate reservoir 452 and for permeate/filtrate reservoir 454, are shown.

The overall workflow for cell growth comprises loading a cell culture to be grown into a first retentate reservoir, optionally bubbling air or an appropriate gas through the cell culture, passing or flowing the cell culture through the first retentate port then tangentially through the TFF channel structure while collecting medium or buffer through one or both of the permeate ports, collecting the cell culture through a second retentate port into a second retentate reservoir, optionally adding additional or different medium to the cell culture and optionally bubbling air or gas through the cell culture, then repeating the process, all while measuring, e.g., the optical density of the cell culture in the retentate reservoirs continuously or at desired intervals. Measurements of optical densities (OD) at programmed time intervals are accomplished using a 600 nm Light Emitting Diode (LED) that has been columnated through an optic into the retentate reservoir(s) containing the growing cells. The light continues through a collection optic to the detection system which consists of a (digital) gain-controlled silicone photodiode. Generally, optical density is shown as the absolute value of the logarithm with base 10 of the power transmission factors of an optical attenuator: OD=-log10 (Power out/Power in). Since OD is the measure of optical attenuation—that is, the sum of absorption, scattering, and reflection—the TFF device OD measurement records the overall power transmission, so as the cells grow and become denser in population, the OD (the loss of signal) increases. The OD system is pre-calibrated against OD standards with these values stored in an on-board memory accessible by the measurement program.

In the channel structure, the membrane bifurcating the flow channels retains the cells on one side of the membrane (the retentate side 422) and allows unwanted medium or buffer to flow across the membrane into a filtrate or permeate side (e.g., permeate member 420) of the device. Bubbling air or other appropriate gas through the cell culture both aerates and mixes the culture to enhance cell growth. During the process, medium that is removed during the flow through the channel structure is removed through the permeate/filtrate ports. Alternatively, cells can be grown in one reservoir with bubbling or agitation without passing the cells through the TFF channel from one reservoir to the other.

The overall workflow for cell concentration using the TFF device/module involves flowing a cell culture or cell sample tangentially through the channel structure. As with the cell growth process, the membrane bifurcating the flow channels retains the cells on one side of the membrane and allows unwanted medium or buffer to flow across the membrane into a permeate/filtrate side (e.g., permeate member 420) of the device. In this process, a fixed volume of cells in medium or buffer is driven through the device until the cell sample is collected into one of the retentate ports, and the medium/buffer that has passed through the membrane is collected through one or both of the permeate/filtrate ports. All types of prokaryotic and eukaryotic cells—both adherent and non-adherent cells—can be grown in the TFF device. Adherent cells may be grown on beads or other cell scaffolds suspended in medium that flow through the TFF device.

The medium or buffer used to suspend the cells in the cell concentration device/module may be any suitable medium or buffer for the type of cells being transformed or transfected, such as LB, SOC, TPD, YPG, YPAD, MEM, DMEM, IMDM, RPMI, Hanks', PBS and Ringer's solution, where the media may be provided in a reagent cartridge as part of a kit. For culture of adherent cells, cells may be disposed on beads, microcarriers, or other type of scaffold suspended in medium. Most normal mammalian tissue-derived cells—except those derived from the hematopoietic system—are anchorage dependent and need a surface or cell culture support for normal proliferation. In the rotating growth vial described herein, microcarrier technology is leveraged. Microcarriers of particular use typically have a diameter of 100-300 μm and have a density slightly greater than that of the culture medium (thus facilitating an easy separation of cells and medium for, e.g., medium exchange) yet the density must also be sufficiently low to allow complete suspension of the carriers at a minimum stirring rate in order to avoid hydrodynamic damage to the cells. Many different types of microcarriers are available, and different microcarriers are optimized for different types of cells. There are positively charged carriers, such as Cytodex 1 (dextran-based, GE Healthcare), DE-52 (cellulose-based, Sigma-Aldrich Labware), DE-53 (cellulose-based, Sigma-Aldrich Labware), and HLX 11-170 (polystyrene-based); collagen- or ECM-(extracellular matrix) coated carriers, such as Cytodex 3 (dextran-based, GE Healthcare) or HyQ-sphere Pro-F 102-4 (polystyrene-based, Thermo Scientific); non-charged carriers, like HyQ-sphere P 102-4 (Thermo Scientific); or macroporous carriers based on gelatin (Cultisphere, Percell Biolytica) or cellulose (Cytopore, GE Healthcare).

In both the cell growth and concentration processes, passing the cell sample through the TFF device and collecting the cells in one of the retentate ports while collecting the medium in one of the permeate/filtrate ports is considered “one pass” of the cell sample. The transfer between retentate reservoirs “flips” the culture. The retentate and permeate ports collecting the cells and medium, respectively, for a given pass reside on the same end of TFF device/module with fluidic connections arranged so that there are two distinct flow layers for the retentate and permeate/filtrate sides, but if the retentate port resides on the retentate member of device/module (that is, the cells are driven through the channel above the membrane and the filtrate (medium) passes to the portion of the channel below the membrane), the permeate/filtrate port will reside on the permeate member of device/module and vice versa (that is, if the cell sample is driven through the channel below the membrane, the filtrate (medium) passes to the portion of the channel above the membrane). Due to the high pressures used to transfer the cell culture and fluids through the flow channel of the TFF device, the effect of gravity is negligible.

At the conclusion of a “pass” in either of the growth and concentration processes, the cell sample is collected by passing through the retentate port and into the retentate reservoir (not shown). To initiate another “pass”, the cell sample is passed again through the TFF device, this time in a flow direction that is reversed from the first pass. The cell sample is collected by passing through the retentate port and into retentate reservoir (not shown) on the opposite end of the device/module from the retentate port that was used to collect cells during the first pass. Likewise, the medium/buffer that passes through the membrane on the second pass is collected through the permeate port on the opposite end of the device/module from the permeate port that was used to collect the filtrate during the first pass, or through both ports. This alternating process of passing the retentate (the concentrated cell sample) through the device/module is repeated until the cells have been grown to a desired optical density, and/or concentrated to a desired volume, and both permeate ports (i.e., if there are more than one) can be open during the passes to reduce operating time. In addition, buffer exchange may be effected by adding a desired buffer (or fresh medium) to the cell sample in the retentate reservoir, before initiating another “pass”, and repeating this process until the old medium or buffer is diluted and filtered out and the cells reside in fresh medium or buffer. Note that buffer exchange and cell growth may (and typically do) take place simultaneously, and buffer exchange and cell concentration may (and typically do) take place simultaneously. For further information and alternative embodiments on TFFs see, e.g., U.S. Ser. No. 16/798,302, filed 22 Feb. 2020.

### The Cell Transformation Module

FIGS. 5A and 5B depict the structure and components of an embodiment of an exemplary reagent cartridge useful in the automated multi-module instrument described therein. In FIG. 5A, reagent cartridge 500 comprises a body 502, which has reservoirs 504. One reservoir 504 is shown empty, and two of the reservoirs have individual tubes (not shown) inserted therein, with individual tube covers 505. Additionally shown are rows of reservoirs into which have been inserted co-joined rows of large tubes 503a, and co-joined rows of small tubes 503b. The co-joined rows of tubes are presented in a strip, with outer flanges 507 that mate on the backside of the outer flange (not shown) with an indentation 509 in the body 502, so as to secure the co-joined rows of tubes (503a and 503b) to the reagent cartridge 500. Shown also is a base 511 of reagent cartridge body 502. Note that the reservoirs 504 in body 502 are shaped generally like the tubes in the co-joined tubes that are inserted into these reservoirs 504.

FIG. 5B depicts the reagent cartridge 500 in FIG. 5A with a row of co-joined large tubes 503a, a row of co-joined small tubes 503b, and one large tube 560 with a cover 505 removed from (i.e., depicted above) the reservoirs 504 of the reagent cartridge 500. Again, the co-joined rows of tubes are presented in a strip, with individual large tubes 561 shown, and individual small tubes 555 shown. Again, each strip of co-joined tubes comprises outer flanges 507 that mate on the backside (not shown) of the outer flange with an indentation 509 in the body 502, to secure the co-joined rows of tubes (503a and 503b) to the reagent cartridge 500. As in FIG. 5A, reagent cartridge body 502 comprises a base 511. Reagent cartridge 500 may be made from any suitable material, including stainless steel, aluminum, or plastics including polyvinyl chloride, cyclic olefin copolymer (COC), polyethylene, polyamide, polypropylene, acrylonitrile butadiene, polycarbonate, polyetheretheketone (PEEK), poly(methyl methylacrylate) (PMMA), polysulfone, and polyurethane, and co-polymers of these and other polymers. Again, if reagent cartridge 500 is disposable, it preferably is made of plastic. In addition, in many embodiments the material used to fabricate the cartridge is thermally-conductive, as reagent cartridge 500 may contact a thermal device (not shown) that heats or cools reagents in the reagent reservoirs 504, including reagents in co-joined tubes. In some embodiments, the thermal device is a Peltier device or thermoelectric cooler.

FIGS. 5C and 5D are top perspective and bottom perspective views, respectively, of an exemplary FTEP device 550 that may be part of (e.g., a component in) reagent cartridge 500 in FIGS. 5A and 5B or may be a stand-alone module; that is, not a part of a reagent cartridge or other module. FIG. 5C depicts an FTEP device 550. The FTEP device 550 has wells that define cell sample inlets 552 and cell sample outlets 554.

FIG. 5D is a bottom perspective view of the FTEP device 550 of FIG. 5C. An inlet well 552 and an outlet well 554 can be seen in this view. Also seen in FIG. 5D are the bottom of an inlet 562 corresponding to well 552, the bottom of an outlet 564 corresponding to the outlet well 554, the bottom of a defined flow channel 566 and the bottom of two electrodes 568 on either side of flow channel 566. The FTEP devices may comprise push-pull pneumatic means to allow multi-pass electroporation procedures; that is, cells to electroporated may be “pulled” from the inlet toward the outlet for one pass of electroporation, then be “pushed” from the outlet end of the FTEP device toward the inlet end to pass between the electrodes again for another pass of electroporation. Further, this process may be repeated one to many times. For additional information regarding FTEP devices, see, e.g., U.S. Pat. Nos. 10,435,713; 10,443,074; 10,323,258; and 10,508,288. Further, other embodiments of the reagent cartridge may provide or accommodate electroporation devices that are not configured as FTEP devices, such as those described in U.S. Ser. No. 16/109,156, filed 22 Aug. 2018. For reagent cartridges useful in the present automated multi-module cell processing instruments, see, e.g., U.S. Pat. Nos. 10,376,889; 10,406,525; 10,576,474; and 10,639,637; and U.S. Ser. No. 16/827,222, filed 23 Mar. 2020.

Additional details of the FTEP devices are illustrated in FIGS. 5E-5G. Note that in the FTEP devices in FIGS. 5E-5G the electrodes are placed such that a first electrode is placed between an inlet and a narrowed region of the flow channel, and the second electrode is placed between the narrowed region of the flow channel and an outlet. FIG. 5E shows a top planar view of an FTEP device 550 having an inlet 552 for introducing a fluid containing cells and exogenous material into FTEP device 550 and an outlet 554 for removing the transformed cells from the FTEP following electroporation. The electrodes 568 are introduced through channels (not shown) in the device.

FIG. 5F shows a cutaway view from the top of the FTEP device 550, with the inlet 552, outlet 554, and electrodes 568 positioned with respect to a flow channel 566. FIG. 5G shows a side cutaway view of FTEP device 550 with the inlet 552 and inlet channel 572, and outlet 554 and outlet channel 574. The electrodes 568 are positioned in electrode channels 576 so that they are in fluid communication with the flow channel 566, but not directly in the path of the cells traveling through the flow channel 566. Note that the first electrode is placed between the inlet and the narrowed region of the flow channel, and the second electrode is placed between the narrowed region of the flow channel and the outlet. The electrodes 568 in this aspect of the device are positioned in the electrode channels 576 which are generally perpendicular to the flow channel 566 such that the fluid containing the cells and exogenous material flows from the inlet channel 572 through the flow channel 566 to the outlet channel 574, and in the process fluid flows into the electrode channels 576 to be in contact with the electrodes 568. In this aspect, the inlet channel, outlet channel and electrode channels all originate from the same planar side of the device. In certain aspects, however, the electrodes may be introduced from a different planar side of the FTEP device than the inlet and outlet channels.

In the FTEP devices of the disclosure, the toxicity level of the transformation results in greater than 30% viable cells after electroporation, preferably greater than 35%, 40%, 45%, 50%, 55%, 60%, 70%, 75%, 80%, 85%, 90%, 95% or even 99% viable cells following transformation, depending on the cell type and the nucleic acids being introduced into the cells.

The housing of the FTEP device can be made from many materials depending on whether the FTEP device is to be reused, autoclaved, or is disposable, including stainless steel, silicon, glass, resin, polyvinyl chloride, polyethylene, polyamide, polystyrene, polyethylene, polypropylene, acrylonitrile butadiene, polycarbonate, polyetheretheketone (PEEK), polysulfone and polyurethane, co-polymers of these and other polymers. Similarly, the walls of the channels in the device can be made of any suitable material including silicone, resin, glass, glass fiber, polyvinyl chloride, polyethylene, polyamide, polyethylene, polypropylene, acrylonitrile butadiene, polycarbonate, polyetheretheketone (PEEK), polysulfone and polyurethane, co-polymers of these and other polymers. Preferred materials include crystal styrene, cyclo-olefin polymer (COP) and cyclic olephin co-polymers (COC), which allow the device to be formed entirely by injection molding in one piece with the exception of the electrodes and, e.g., a bottom sealing film if present.

The FTEP devices described herein (or portions of the FTEP devices) can be created or fabricated via various techniques, e.g., as entire devices or by creation of structural layers that are fused or otherwise coupled. For example, for metal FTEP devices, fabrication may include precision mechanical machining or laser machining; for silicon FTEP devices, fabrication may include dry or wet etching; for glass FTEP devices, fabrication may include dry or wet etching, powderblasting, sandblasting, or photostructuring; and for plastic FTEP devices fabrication may include thermoforming, injection molding, hot embossing, or laser machining.

The components of the FTEP devices may be manufactured separately and then assembled, or certain components of the FTEP devices (or even the entire FTEP device except for the electrodes) may be manufactured (e.g., using 3D printing) or molded (e.g., using injection molding) as a single entity, with other components added after molding. For example, housing and channels may be manufactured or molded as a single entity, with the electrodes later added to form the FTEP unit. Alternatively, the FTEP device may also be formed in two or more parallel layers, e.g., a layer with the horizontal channel and filter, a layer with the vertical channels, and a layer with the inlet and outlet ports, which are manufactured and/or molded individually and assembled following manufacture.

In specific aspects, the FTEP device can be manufactured using a circuit board as a base, with the electrodes, filter and/or the flow channel formed in the desired configuration on the circuit board, and the remaining housing of the device containing, e.g., the one or more inlet and outlet channels and/or the flow channel formed as a separate layer that is then sealed onto the circuit board. The sealing of the top of the housing onto the circuit board provides the desired configuration of the different elements of the FTEP devices of the disclosure. Also, two to many FTEP devices may be manufactured on a single substrate, then separated from one another thereafter or used in parallel. In certain embodiments, the FTEP devices are reusable and, in some embodiments, the FTEP devices are disposable. In additional embodiments, the FTEP devices may be autoclavable.

The electrodes 568 can be formed from any suitable metal, such as copper, stainless steel, titanium, aluminum, brass, silver, rhodium, gold or platinum, or graphite. One preferred electrode material is alloy 303 (UNS330300) austenitic stainless steel. An applied electric field can destroy electrodes made from of metals like aluminum. If a multiple-use (i.e., non-disposable) flow-through FTEP device is desired—as opposed to a disposable, one-use flow-through FTEP device—the electrode plates can be coated with metals resistant to electrochemical corrosion. Conductive coatings like noble metals, e.g., gold, can be used to protect the electrode plates.

As mentioned, the FTEP devices may comprise push-pull pneumatic means to allow multi-pass electroporation procedures; that is, cells to be electroporated may be “pulled” from the inlet toward the outlet for one pass of electroporation, then be “pushed” from the outlet end of the flow-through FTEP device toward the inlet end to pass between the electrodes again for another pass of electroporation. This process may be repeated one to many times.

Depending on the type of cells to be electroporated (e.g., bacterial, yeast, mammalian) and the configuration of the electrodes, the distance between the electrodes in the flow channel can vary widely. For example, where the flow channel decreases in width, the flow channel may narrow to between 10 μm and 5 mm, or between 25 μm and 3 mm, or between 50 μm and 2 mm, or between 75 μm and 1 mm. The distance between the electrodes in the flow channel may be between 1 mm and 10 mm, or between 2 mm and 8 mm, or between 3 mm and 7 mm, or between 4 mm and 6 mm. The overall size of the FTEP device may be from 3 cm to 15 cm in length, or 4 cm to 12 cm in length, or 4.5 cm to 10 cm in length. The overall width of the FTEP device may be from 0.5 cm to 5 cm, or from 0.75 cm to 3 cm, or from 1 cm to 2.5 cm, or from 1 cm to 1.5 cm.

The region of the flow channel that is narrowed is wide enough so that at least two cells can fit in the narrowed portion side-by-side. For example, a typical bacterial cell is 1 μm in diameter; thus, the narrowed portion of the flow channel of the FTEP device used to transform such bacterial cells will be at least 2 μm wide. In another example, if a mammalian cell is approximately 50 μm in diameter, the narrowed portion of the flow channel of the FTEP device used to transform such mammalian cells will be at least 100 μm wide. That is, the narrowed portion of the FTEP device will not physically contort or “squeeze” the cells being transformed.

In embodiments of the FTEP device where reservoirs are used to introduce cells and exogenous material into the FTEP device, the reservoirs range in volume from 100 μL to 10 mL, or from 500 μL to 75 mL, or from 1 mL to 5 mL. The flow rate in the FTEP ranges from 0.1 mL to 5 mL per minute, or from 0.5 mL to 3 mL per minute, or from 1.0 mL to 2.5 mL per minute. The pressure in the FTEP device ranges from 1-30 psi, or from 2-10 psi, or from 3-5 psi.

To avoid different field intensities between the electrodes, the electrodes should be arranged in parallel. Furthermore, the surface of the electrodes should be as smooth as possible without pin holes or peaks. Electrodes having a roughness Rz of 1 to 10 μm are preferred. In another embodiment of the invention, the flow-through electroporation device comprises at least one additional electrode which applies a ground potential to the FTEP device. Flow-through electroporation devices (either as a stand-alone instrument or as a module in an automated multi-module system) are described in, e.g., U.S. Pat. No. 10,435,713, issued 8 Oct. 2019; U.S. Pat. No. 10,443,074, issued 15 Oct. 2019; U.S. Pat. No. 10,323,258, issued 30 Sep. 2019; U.S. Pat. No. 10,508,288, issued 17 Dec. 2019; U.S. Pat. No. 10,415,058, issued 17 Sep. 2019; and U.S. Pat. No. 10,557,150, issued 11 Feb. 2020; and U.S. Ser. No. 16/550,790, filed 26 Aug. 2019; and Ser. No. 16/548,208, filed 22 Aug. 2019.

### Cell Singulation and Enrichment Device

In some embodiments, after transformation and prior to editing, transformed cells are “singulated” or isolated in, e.g., wells. As described supra, singulating or isolating transformed cells allows each cell to grow into a clonal colony without competition from other cells. FIG. 6A and the accompanying description thereof describes this workflow and the advantages thereof. FIG. 6A depicts a solid wall device 6050 and a workflow for singulating cells in microwells in the solid wall device. At the top left of the figure (i), there is depicted solid wall device 6050 with microwells 6052. A section 6054 of substrate 6050 is shown at (ii), also depicting microwells 6052. At (iii), a side cross-section of solid wall device 6050 is shown, and microwells 6052 have been loaded, where, in this embodiment, Poisson or substantial Poisson loading has taken place; that is, each microwell has one or no cells, and the likelihood that any one microwell has more than one cell is low. At (iv), workflow 6040 is illustrated where substrate 6050 having microwells 6052 shows microwells 6056 with one cell per microwell, microwells 6057 with no cells in the microwells, and one microwell 6060 with two cells in the microwell. In step 6051, the cells in the microwells are allowed to double approximately 2-150 times to form clonal colonies (v), then editing is allowed to occur 6053. For growth and editing, the medium used will depend, of course, on the type of cells being edited—e.g., bacterial, yeast or mammalian. For example, medium for mammalian cell growth includes MEM, DMEM, IMDM, RPMI, Hanks', PBS and Ringer's solution.

After editing 6053, many cells in the colonies of cells that have been edited die as a result of the double-strand cuts caused by active editing and there is a lag in growth for the edited cells that do survive but must repair and recover following editing (microwells 6058), where cells that do not undergo editing thrive (microwells 6059) (vi). All cells are allowed to continue grow to establish colonies and normalize, where the colonies of edited cells in microwells 6058 catch up in size and/or cell number with the cells in microwells 6059 that do not undergo editing (vii). Once the cell colonies are normalized, the cells are lysed, pooled and sequenced 6060, the nucleic acids are treated to bisulfite conversion 6061 as described supra, and the editing cassettes are then correlated with edits to the target genome.

A module useful for performing the methods depicted in FIG. 6A is a solid wall isolation, incubation, and normalization (SWIIN) module. FIG. 6B depicts an embodiment of a SWIIN module 650 from an exploded top perspective view. In SWIIN module 650 the retentate member is formed on the bottom of a top of a SWIIN module component and the permeate member is formed on the top of the bottom of a SWIIN module component.

The SWIIN module 650 in FIG. 6B comprises from the top down, a reservoir gasket or cover 658, a retentate member 604 (where a retentate flow channel cannot be seen in this FIG. 6C), a perforated member 601 swaged with a filter (filter not seen in FIG. 6B), a permeate member 608 comprising integrated reservoirs (permeate reservoirs 652 and retentate reservoirs 654), and two reservoir seals 662, which seal the bottom of permeate reservoirs 652 and retentate reservoirs 654. A permeate channel 660a can be seen disposed on the top of permeate member 608, defined by a raised portion 676 of serpentine channel 660a, and ultrasonic tabs 664 (see FIG. 6C) can be seen disposed on the top of permeate member 608 as well. The perforations that form the wells on perforated member 601 are not seen in this FIG. 6B; however, through-holes 666 to accommodate the ultrasonic tabs 664 are seen. In addition, supports 670 are disposed at either end of SWIIN module 650 to support SWIIN module 650 and to elevate permeate member 608 and retentate member 604 above reservoirs 652 and 654 to minimize bubbles or air entering the fluid path from the permeate reservoir to serpentine channel 660a or the fluid path from the retentate reservoir to serpentine channel 660b (neither fluid path is seen in this FIG. 6B).

In this FIG. 6B, it can be seen that the serpentine channel 660a that is disposed on the top of permeate member 608 traverses permeate member 608 for most of the length of permeate member 608 except for the portion of permeate member 608 that comprises permeate reservoirs 652 and retentate reservoirs 654 and for most of the width of permeate member 608. As used herein with respect to the distribution channels in the retentate member or permeate member, “most of the length” means about 95% of the length of the retentate member or permeate member, or about 90%, 85%, 80%, 75%, or 70% of the length of the retentate member or permeate member. As used herein with respect to the distribution channels in the retentate member or permeate member, “most of the width” means about 95% of the width of the retentate member or permeate member, or about 90%, 85%, 80%, 75%, or 70% of the width of the retentate member or permeate member.

In this embodiment of a SWIIN module, the perforated member includes through-holes to accommodate ultrasonic tabs disposed on the permeate member. Thus, in this embodiment the perforated member is fabricated from 316 stainless steel, and the perforations form the walls of microwells while a filter or membrane is used to form the bottom of the microwells. Typically, the perforations (microwells) are approximately 150 μm-200 μm in diameter, and the perforated member is approximately 125 μm deep, resulting in microwells having a volume of approximately 2.5 nL, with a total of approximately 200,000 microwells. The distance between the microwells is approximately 279 μm center-to-center. Though here the microwells have a volume of approximately 2.5 nL, the volume of the microwells may be from 1 to 25 nL, or preferably from 2 to 10 nL, and even more preferably from 2 to 4 nL. As for the filter or membrane, like the filter described previously, filters appropriate for use are solvent resistant, contamination free during filtration, and are able to retain the types and sizes of cells of interest. For example, in order to retain small cell types such as bacterial cells, pore sizes can be as low as 0.10 μm, however for other cell types (e.g., such as for mammalian cells), the pore sizes can be as high as 10.0 μm-20.0 μm or more. Indeed, the pore sizes useful in the cell concentration device/module include filters with sizes from 0.10 μm, 0.11 μm, 0.12 μm, 0.13 μm, 0.14 μm, 0.15 μm, 0.16 μm, 0.17 μm, 0.18 μm, 0.19 μm, 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.36 μm, 0.37 μm, 0.38 μm, 0.39 μm, 0.40 μm, 0.41 μm, 0.42 μm, 0.43 μm, 0.44 μm, 0.45 μm, 0.46 μm, 0.47 μm, 0.48 μm, 0.49 μm, 0.50 μm and larger. The filters may be fabricated from any suitable material including cellulose mixed ester (cellulose nitrate and acetate) (CME), polycarbonate (PC), polyvinylidene fluoride (PVDF), polyethersulfone (PES), polytetrafluoroethylene (PTFE), nylon, or glass fiber.

The cross-section configuration of the mated serpentine channel may be round, elliptical, oval, square, rectangular, trapezoidal, or irregular. If square, rectangular, or another shape with generally straight sides, the cross section may be from about 2 mm to 15 mm wide, or from 3 mm to 12 mm wide, or from 5 mm to 10 mm wide. If the cross section of the mated serpentine channel is generally round, oval or elliptical, the radius of the channel may be from about 3 mm to 20 mm in hydraulic radius, or from 5 mm to 15 mm in hydraulic radius, or from 8 mm to 12 mm in hydraulic radius.

Serpentine channels 660a and 660b can have approximately the same volume or a different volume. For example, each “side” or portion 660a, 660b of the serpentine channel may have a volume of, e.g., 2 mL, or serpentine channel 660a of permeate member 608 may have a volume of 2 mL, and the serpentine channel 660b of retentate member 604 may have a volume of, e.g., 3 mL. The volume of fluid in the serpentine channel may range from about 2 mL to about 80 mL, or about 4 mL to 60 mL, or from 5 mL to 40 mL, or from 6 mL to 20 mL (note these volumes apply to a SWIIN module comprising a, e.g., 50-500K perforation member). The volume of the reservoirs may range from 5 mL to 50 mL, or from 7 mL to 40 mL, or from 8 mL to 30 mL or from 10 mL to 20 mL, and the volumes of all reservoirs may be the same or the volumes of the reservoirs may differ (e.g., the volume of the permeate reservoirs is greater than that of the retentate reservoirs).

The serpentine channel portions 660a and 660b of the permeate member 608 and retentate member 604, respectively, are approximately 200 mm long, 130 mm wide, and 4 mm thick, though in other embodiments, the retentate and permeate members can be from 75 mm to 400 mm in length, or from 100 mm to 300 mm in length, or from 150 mm to 250 mm in length; from 50 mm to 250 mm in width, or from 75 mm to 200 mm in width, or from 100 mm to 150 mm in width; and from 2 mm to 15 mm in thickness, or from 4 mm to 10 mm in thickness, or from 5 mm to 8 mm in thickness. Embodiments the retentate (and permeate) members may be fabricated from PMMA (poly(methyl methacrylate) or other materials may be used, including polycarbonate, cyclic olefin co-polymer (COC), glass, polyvinyl chloride, polyethylene, polyamide, polypropylene, polysulfone, polyurethane, and co-polymers of these and other polymers. Preferably at least the retentate member is fabricated from a transparent material so that the cells can be visualized (see, e.g., FIG. 6E and the description thereof). For example, a video camera may be used to monitor cell growth by, e.g., density change measurements based on an image of an empty well, with phase contrast, or if, e.g., a chromogenic marker, such as a chromogenic protein, is used to add a distinguishable color to the cells. Chromogenic markers such as blitzen blue, dreidel teal, virginia violet, vixen purple, prancer purple, tinsel purple, maccabee purple, donner magenta, cupid pink, seraphina pink, scrooge orange, and leor orange (the Chromogenic Protein Paintbox, all available from ATUM (Newark, Calif.)) obviate the need to use fluorescence, although fluorescent cell markers, fluorescent proteins, and chemiluminescent cell markers may also be used.

Because the retentate member preferably is transparent, colony growth in the SWIIN module can be monitored by automated devices such as those sold by JoVE (ScanLag™ system, Cambridge, Mass.) (also see Levin-Reisman, et al., Nature Methods, 7:737-39 (2010)). Cell growth for, e.g., mammalian cells may be monitored by, e.g., the growth monitor sold by IncuCyte (Ann Arbor, Mich.) (see also, Choudhry, PLOS ONE, 11(2):e0148469 (2016)). Further, automated colony pickers may be employed, such as those sold by, e.g., TECAN (Pickolo™ system, Mannedorf, Switzerland); Hudson Inc. (RapidPick™, Springfield, N.J.); Molecular Devices (QPix 400™ system, San Jose, Calif.); and Singer Instruments (PIXL™ system, Somerset, UK).

Due to the heating and cooling of the SWIIN module, condensation may accumulate on the retentate member which may interfere with accurate visualization of the growing cell colonies. Condensation of the SWIIN module 650 may be controlled by, e.g., moving heated air over the top of (e.g., retentate member) of the SWIIN module 650, or by applying a transparent heated lid over at least the serpentine channel portion 660b of the retentate member 604. See, e.g., FIG. 6E and the description thereof infra.

In SWIIN module 650 cells and medium—at a dilution appropriate for Poisson or substantial Poisson distribution of the cells in the microwells of the perforated member—are flowed into serpentine channel 660b from ports in retentate member 604, and the cells settle in the microwells while the medium passes through the filter into serpentine channel 660a in permeate member 608. The cells are retained in the microwells of perforated member 601 as the cells cannot travel through filter 603. Appropriate medium may be introduced into permeate member 608 through the permeate ports. The medium flows upward through filter (not shown but beneath and adjacent to perforated member 601) to nourish the cells in the microwells (perforations) of perforated member 601. Additionally, buffer exchange can be effected by cycling medium through the retentate and permeate members. In operation, the cells are deposited into the microwells, are grown for an initial, e.g., 2-100 doublings, editing is induced by, e.g., raising the temperature of the SWIIN to 42° C. to induce a temperature inducible promoter or by removing growth medium from the permeate member and replacing the growth medium with a medium comprising a chemical component that induces an inducible promoter.

Once editing has taken place, the temperature of the SWIIN may be decreased, or the inducing medium may be removed and replaced with fresh medium lacking the chemical component thereby de-activating the inducible promoter. The cells then continue to grow in the SWIIN module 650 until the growth of the cell colonies in the microwells is normalized. For the normalization protocol, once the colonies are normalized, the colonies are flushed from the microwells by applying fluid or air pressure (or both) to the permeate member serpentine channel 660a and thus to filter (not shown but beneath and adjacent to perforated member 601) and pooled. Alternatively, if cherry picking is desired, the growth of the cell colonies in the microwells is monitored, and slow-growing colonies are directly selected; or, fast-growing colonies are eliminated.

FIG. 6C is a top perspective view of a SWIIN module with the retentate and perforated members in partial cross section. In this FIG. 6C, it can be seen that serpentine channel 660a is disposed on the top of permeate member 608 is defined by raised portions 676 and traverses permeate member 608 for most of the length and width of permeate member 608 except for the portion of permeate member 608 that comprises the permeate and retentate reservoirs (note only one retentate reservoir 652 can be seen). Moving from left to right, reservoir gasket 658 is disposed upon the integrated reservoir cover 678 (cover not seen in this FIG. 6C) of retentate member 604. Gasket 658 comprises reservoir access apertures 632a, 632b, 632c, and 632d, as well as pneumatic ports 633a, 633b, 633c and 633d. Also at the far left end is support 670. Disposed under permeate reservoir 652 can be seen one of two reservoir seals 662. In addition to the retentate member being in cross section, the perforated member 601 and filter 603 (filter 603 is not seen in this FIG. 6C) are in cross section. Note that there are a number of ultrasonic tabs 664 disposed at the right end of SWIIN module 650 and on raised portion 676 which defines the channel turns of serpentine channel 660a, including ultrasonic tabs 664 extending through through-holes (not seen here but see 666 in FIG. 6B) of perforated member 601. There is also a support 670 at the end distal reservoirs 652, 654 of permeate member 608.

FIG. 6D is a side perspective view of an assembled SWIIIN module 650, including, from right to left, reservoir gasket 658 disposed upon integrated reservoir cover 678 (not seen) of retentate member 604. Gasket 658 may be fabricated from rubber, silicone, nitrile rubber, polytetrafluoroethylene, a plastic polymer such as polychlorotrifluoroethylene, or other flexible, compressible material. Gasket 658 comprises reservoir access apertures 632a, 632b, 632c, and 632d, as well as pneumatic ports 633a, 633b, 633c and 633d. Also at the far-left end is support 670 of permeate member 608. In addition, permeate reservoir 652 can be seen, as well as one reservoir seal 662. At the far-right end is a second support 670.

Imaging of cell colonies growing in the wells of the SWIIN is desired in most implementations for, e.g., monitoring both cell growth and device performance. Real-time monitoring of cell growth in the SWIIN requires backlighting, retentate plate (top plate) condensation management and a system-level approach to temperature control, air flow, and thermal management. In some implementations, imaging employs a camera or CCD device with sufficient resolution to be able to image individual wells. For example, in some configurations a camera with a 9-pixel pitch is used (that is, there are 9 pixels center-to-center for each well). Processing the images may, in some implementations, utilize reading the images in grayscale, rating each pixel from low to high, where wells with no cells will be brightest (due to full or nearly-full light transmission from the backlight) and wells with cells will be dim (due to cells blocking light transmission from the backlight).

After processing the images, thresholding is performed to determine which pixels will be called “bright” or “dim”, spot finding is performed to find bright pixels and arrange them into blocks, and then the spots are arranged on a hexagonal grid of pixels that correspond to the spots. Once arranged, the measure of intensity of each well is extracted, by, e.g., looking at one or more pixels in the middle of the spot, looking at several to many pixels at random or pre-set positions, or averaging X number of pixels in the spot. In addition, background intensity may be subtracted. Thresholding is again used to call each well positive (e.g., containing cells) or negative (e.g., no cells in the well). The imaging information may be used in several ways, including taking images at time points for monitoring cell growth. Monitoring cell growth can be used to, e.g., remove the “muffin tops” of fast-growing cells followed by removal of all cells or removal of cells in “rounds” as described above, or recover cells from specific wells (e.g., slow-growing cell colonies); alternatively, wells containing fast-growing cells can be identified and areas of UV light covering the fast-growing cell colonies can be projected (or rastered with shutters) onto the SWIIN to irradiate or inhibit growth of those cells. Imaging may also be used to assure proper fluid flow in the serpentine channel 660.

FIG. 6E depicts the embodiment of the SWIIN module in FIGS. 6B-6E further comprising a heat management system including a heater and a heated cover. The heater cover facilitates the condensation management that is required for imaging. Assembly 698 comprises a SWIIN module 650 seen lengthwise in cross section, where one permeate reservoir 652 is seen. Disposed immediately upon SWIIN module 650 is cover 694 and disposed immediately below SWIIN module 650 is backlight 680, which allows for imaging. Beneath and adjacent to the backlight and SWIIN module is insulation 682, which is disposed over a heatsink 684. In this FIG. 6F, the fins of the heatsink would be in-out of the page. In addition there is also axial fan 686 and heat sink 688, as well as two thermoelectric coolers 692, and a controller 690 to control the pneumatics, thermoelectric coolers, fan, solenoid valves, etc. The arrows denote cool air coming into the unit and hot air being removed from the unit. It should be noted that control of heating allows for growth of many different types of cells (prokaryotic and eukaryotic) as well as strains of cells that are, e.g., temperature sensitive, etc., and allows use of temperature-sensitive promoters. Temperature control allows for protocols to be adjusted to account for differences in transformation efficiency, cell growth and viability. For more details regarding solid wall isolation incubation and normalization devices and methods see U.S. Pat. No. 10,533,152, issued 14 Jan. 2020; U.S. Pat. No. 10,532,324, issued 14 Jan. 2020; U.S. Pat. No. 10,550,363, issued 4 Feb. 2020; U.S. Pat. No. 10,625,212, issued 21 Apr. 2020; U.S. Pat. No. 10,663,626, issued 28 Apr. 2020; U.S. Pat. No. 10,633,627, issued 28 Apr. 2020; U.S. Pat. No. 10,647,958, issued 12 May 2020; and U.S. Ser. No. 16/823,269, filed 18 Mar. 2020; Ser. No. 16/820,292, filed 16 Mar. 2020; Ser. No. 16/844,339, filed 9 Apr. 2020; Ser. No. 16/686,066, filed 15 Nov. 2019; and Ser. No. 16/820,324, filed 16 Mar. 2020.

It should be apparent to one of ordinary skill in the art given the present disclosure that the processes described may be recursive and multiplexed; that is, cells may go through the workflow described in relation to FIG. 1A, then the resulting edited population of cells may go through another (or several or many) rounds of additional editing (e.g., recursive editing) with different editing vectors. For example, the cells from round 1 of editing may be diluted and an aliquot of the edited cells edited by editing vector A may be combined with editing vector B, an aliquot of the edited cells edited by editing vector A may be combined with editing vector C, an aliquot of the edited cells edited by editing vector A may be combined with editing vector D, and so on for a second round of editing. After round two, an aliquot of each of the double-edited cells may be subjected to a third round of editing, where, e.g., aliquots of each of the AB-, AC-, AD-edited cells are combined with additional editing vectors, such as editing vectors X, Y, and Z. That is that double-edited cells AB may be combined with and edited by vectors X, Y, and Z to produce triple-edited edited cells ABX, ABY, and ABZ; double-edited cells AC may be combined with and edited by vectors X, Y, and Z to produce triple-edited cells ACX, ACY, and ACZ; and double-edited cells AD may be combined with and edited by vectors X, Y, and Z to produce triple-edited cells ADX, ADY, and ADZ, and so on. In this process, many permutations and combinations of edits can be executed, leading to very diverse cell populations and cell libraries. In any recursive process, it is advantageous to “cure” the previous engine and editing vectors (or single engine+editing vector in a single vector system).

### Bioreactor

In addition to the rotating growth vial module shown in FIGS. 3A-3E and described in the related text, and the tangential flow filtration (TFF) module shown FIG. 4A-4G and described in the related text, a bioreactor can be used to grow cells off-instrument or to allow for cell growth and recovery on-instrument; e.g., as one module of the multi-module fully-automated closed instrument. Further, the bioreactor supports cell selection/enrichment, via expressed antibiotic markers in the growth process or via expressed antibodies coupled to magnetic beads and a magnet associated with the bioreactor. There are many bioreactors known in the art, including those described in, e.g., WO 2019/046766; U.S. Pat. Nos. 10,699,519; 10,633,625; 10,577,576; 10,294,447; 10,240,117; 10,179,898; 10,370,629; and 9,175,259; and those available from Lonza Group Ltd. (Basel, Switzerland); Miltenyi Biotec (Bergisch Gladbach, Germany), Terumo BCT (Lakewood, Colo.) and Sartorius GmbH (Gottingen, Germany).

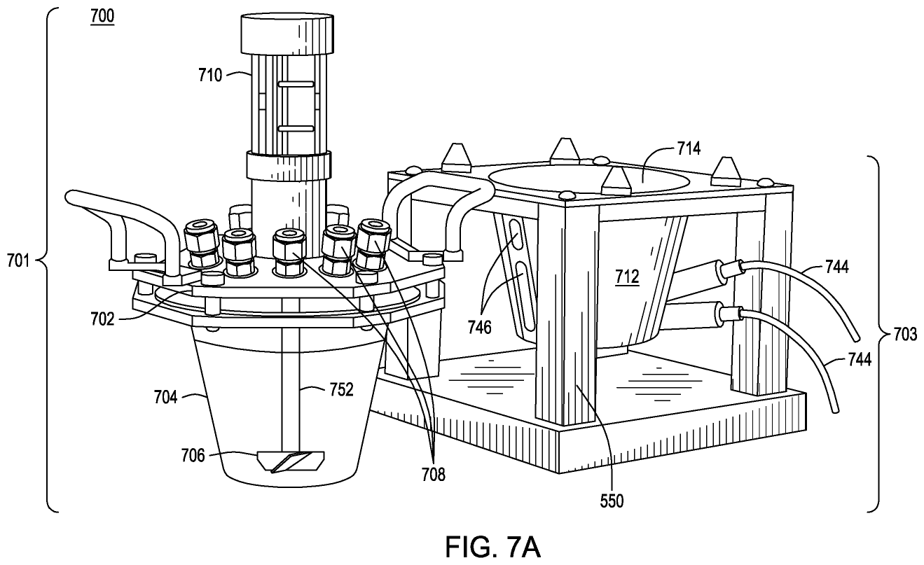

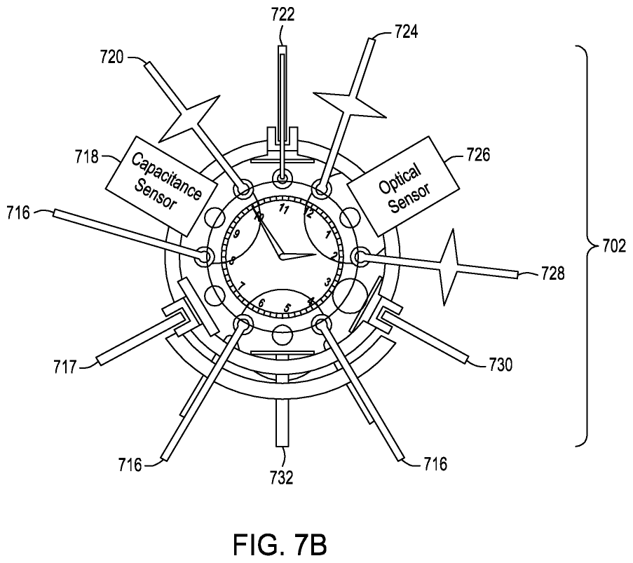

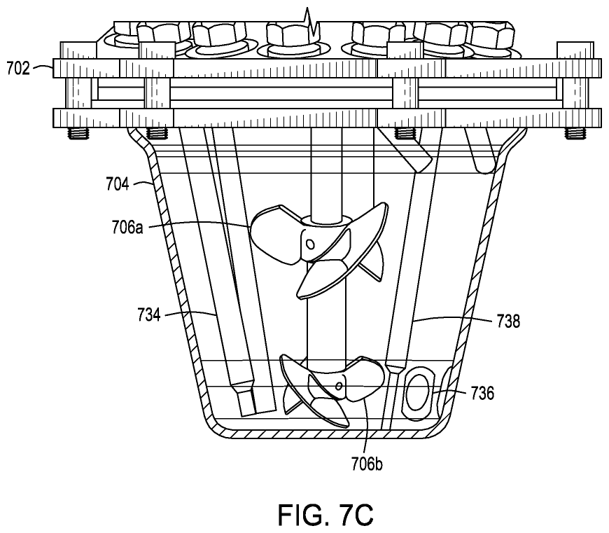

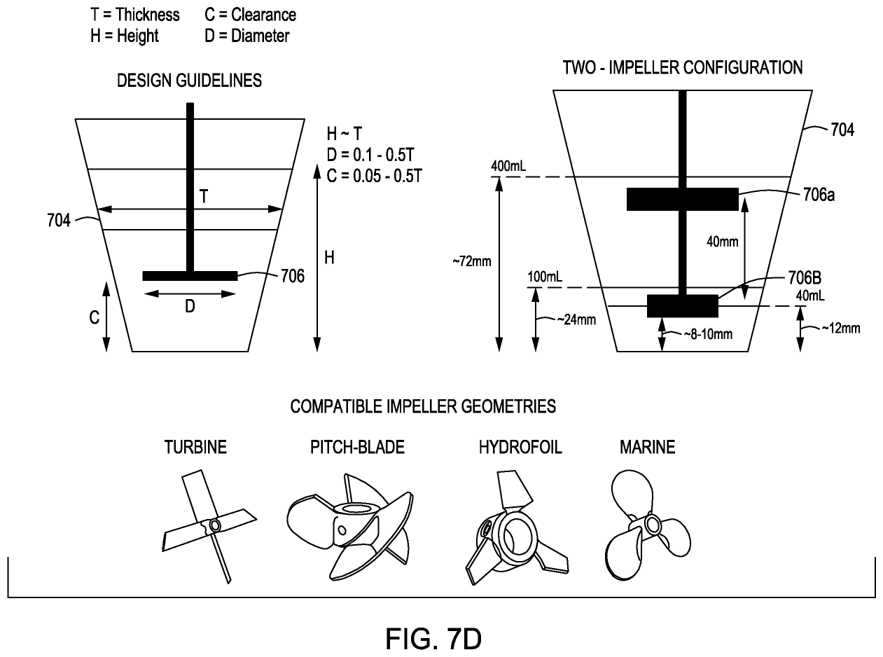

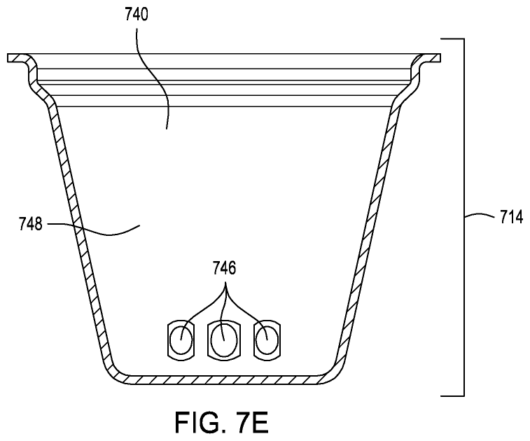

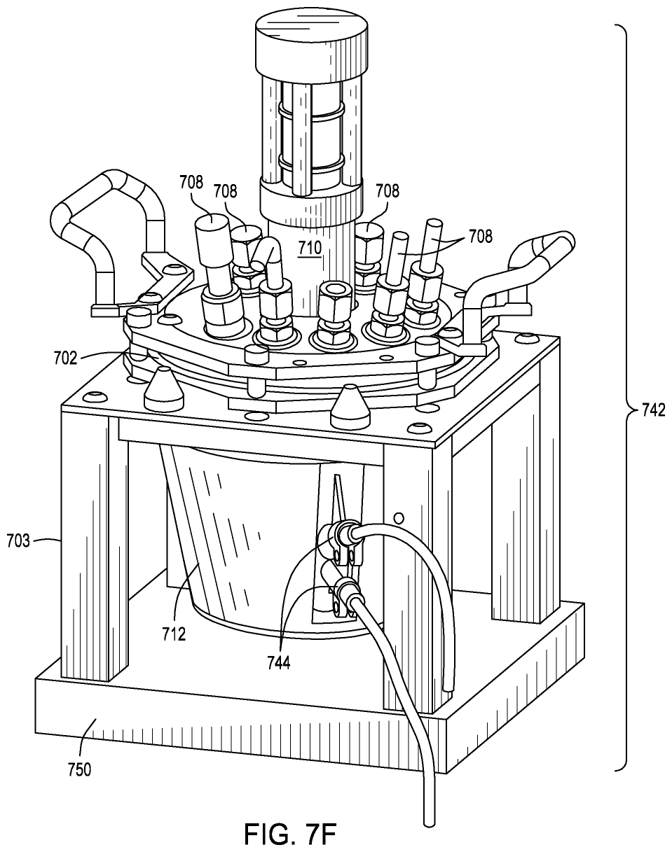

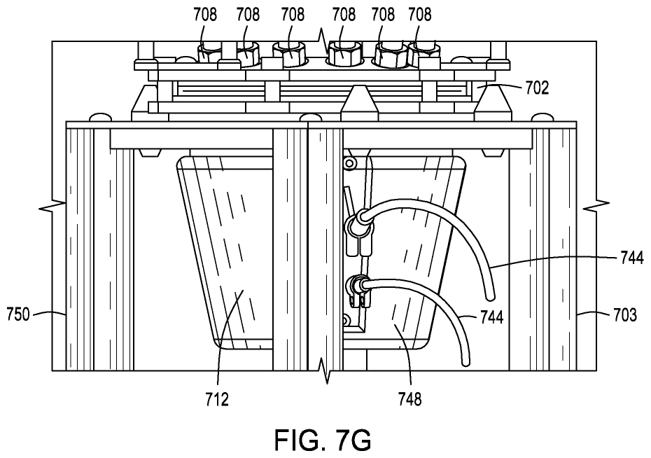

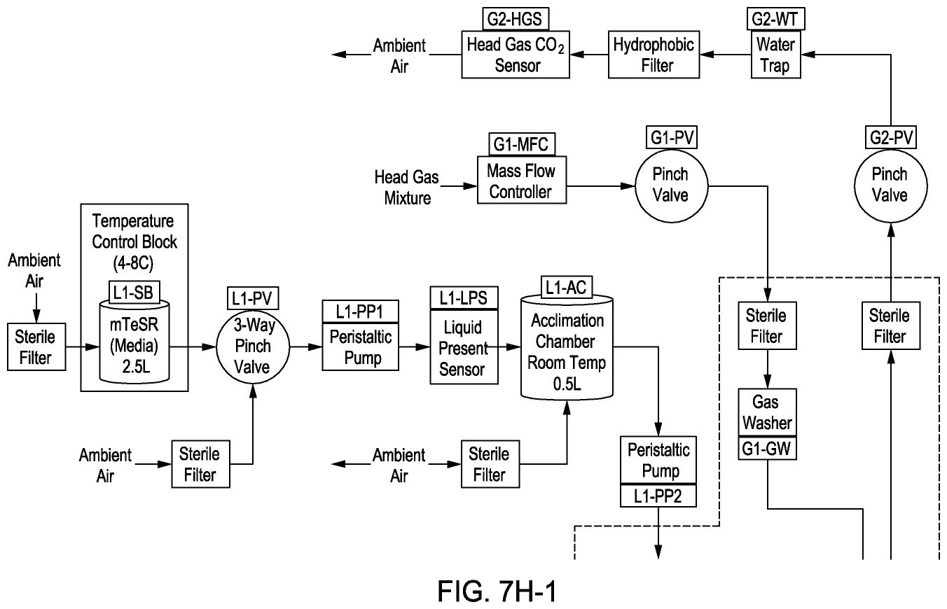

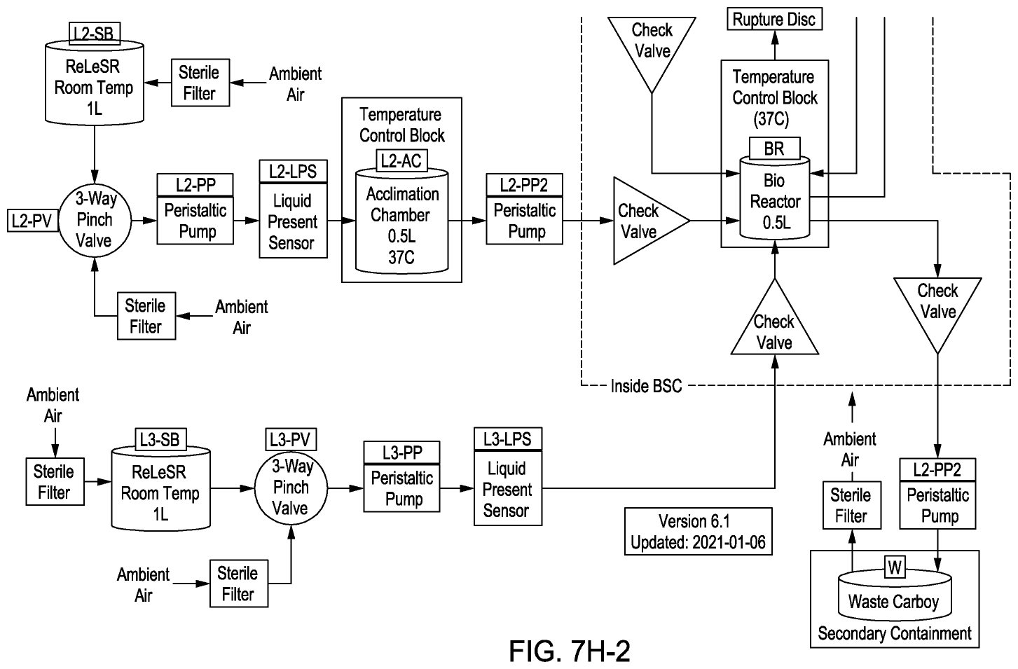

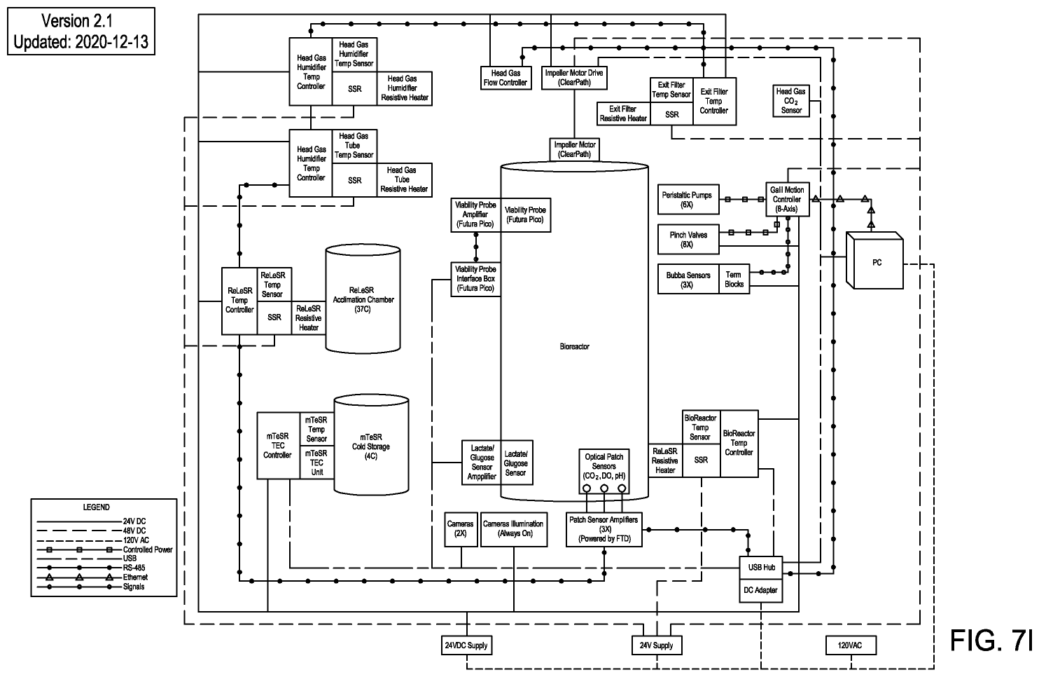

FIG. 7A shows one embodiment of a bioreactor assembly 700 for cell growth, transfection, and editing in the automated multi-module cell processing instruments described herein. Unlike most bioreactors that are used to support fermentation or other processes with an eye to harvesting the products produced by organisms grown in the bioreactor, the present bioreactor (and the processes performed therein) is configured to grow cells, monitor cell growth (via, e.g., optical means or capacitance), passage cells, select cells, transfect cells, and support the growth and harvesting of edited cells. Bioreactor assembly 700 comprises cell growth, transfection, and editing vessel 701 comprising a main body 704 with a lid assembly 702 comprising ports 708, including an optional motor integration port 710 driving impeller 706 via impeller shaft 752. Bioreactor assembly 700 comprises a growth vessel 701 comprising tapered a main body 704 with a lid assembly 702 comprising ports 708, including an optional motor integration port 710 driving impeller 706 via impeller shaft 752. The tapered shape of main body 704 of the vessel 701 along with, in some embodiments, dual impellers allows for working with a larger dynamic range of volumes, such as, e.g., up to 700 ml and as low as 100 ml for rapid sedimentation of the microcarriers. In addition, the low volume is useful for magnetic bead separation or enrichment as described above.

Bioreactor assembly 700 further comprises bioreactor stand assembly 703 comprising a main body 712 and vessel holder 714 comprising a heat jacket or other heating means (not shown, but see FIG. 7E) into which the main body 704 of vessel 701 is disposed in operation. The main body 704 of vessel 701 is biocompatible and preferably transparent—in some embodiments, in the UV and IR range as well as the visible spectrum—so that the growing cells can be visualized by, e.g., cameras or sensors integrated into lid assembly 702 or through viewing apertures or slots in the main body 712 of bioreactor stand assembly 703 (not shown in this FIG. 7A, but see FIG. 7E).

Bioreactor assembly 700 supports growth of cells from a 500,000 cell input to a 10 billion cell output, or from a 1 million cell input to a 25 billion cell output, or from a 5 million cell input to a 50 billion cell output or combinations of these ranges depending on, e.g., the size of main body 704 of vessel 701, the medium used to grow the cells, whether the cells are adherent or non-adherent. The bioreactor that comprises assembly 700 supports growth of both adherent and non-adherent cells, wherein adherent cells are typically grown of microcarriers as described in detail above and supra or as spheroids. Alternatively, another option for growing mammalian cells in the bioreactor described herein is growing single cells in suspension using a specialized medium such as that developed by ACCELLTA™ (Haifa, Israel). Cells grown in this medium must be adapted to this process over many cell passages; however, once adapted the cells can be grown to a density of >40 million cells/ml and expanded 50-100× in approximately a week, depending on cell type.

Main body 704 of vessel 701 preferably is manufactured by injection molding, as is, in some embodiments, impeller 706 and the impeller shaft (not shown). Impeller 706 also may be fabricated from stainless steel, metal, plastics or the polymers listed infra. Injection molding allows for flexibility in size and configuration and also allows for, e.g., volume markings to be added to the main body 704 of vessel 701. Additionally, material from which the main body 704 of vessel 701 is fabricated should be able to be cooled to about 4° C. or lower and heated to about 55° C. or higher to accommodate cell growth. Further, the material that is used to fabricate the vial preferably is able to withstand temperatures up to 55° C. without deformation. Suitable materials for main body 704 of vessel 701 include those described for the rotating growth vial described in relation to FIGS. 3A and 3B and the TFF device described in relation to FIG. 4A-4E, including cyclic olefin copolymer (COC), glass, polyvinyl chloride, polyethylene, polyetheretherketone (PEEK), polypropylene, polycarbonate, poly(methyl methacrylate (PMMA)), polysulfone, poly(dimethylsiloxane), cyclo-olefin polymer (COP), and co-polymers of these and other polymers. Preferred materials include polypropylene, polycarbonate, or polystyrene. The material used for fabrication may depend on the cell type to be grown, transfected and edited, and is conducive to growth of both adherent and non-adherent cells and workflows involving microcarrier-based transfection. The main body 704 of vessel 701 may be reusable or, alternatively, may be manufactured and configured for a single use. In one embodiment, main body 704 of vessel 701 may support cell culture volumes of 25 ml to 500 ml, but may be scaled up to support cell culture volumes of up to 3 L.

The bioreactor stand assembly comprises a stand or frame 750, a main body 712 which holds the vessel 701 during operation. The stand/frame 750 and main body 712 are fabricated from stainless steel, other metals, or polymer/plastics. The bioreactor main body further comprises a heat jacket (not seen in FIG. 7A, but see FIG. 7E) to maintain the bioreactor main body 704—and thus the cell culture—at a desired temperature. Essentially, the stand assembly can host a set of sensors and cameras to monitor cell culture.

FIG. 7B depicts a top-down view of one embodiment of vessel lid assembly 702. Vessel lid assembly 702 is configured to be air-tight, providing a sealed, sterile environment for cell growth, transfection and editing as well as to provide biosafety maintaining a closed system. Vessel lid assembly 702 and the main body 704 of vessel 701 can be sealed via fasteners such as screws, using biocompatible glues, or the two components may be ultrasonically welded. Vessel lid assembly 702 is some embodiments is fabricated from stainless steel such as S316L stainless steel but may also be fabricated from metals, other polymers (such as those listed supra) or plastics. As seen in this FIG. 7B—as well as in FIG. 7A—vessel lid assembly 702 comprises a number of different ports to accommodate liquid addition and removal; gas addition and removal; for insertion of sensors to monitor culture parameters (described in more detail infra); to accommodate one or more cameras or other optical sensors; to provide access to the main body 704 of vessel 701 by, e.g., a liquid handling device (see FIG. 7A for main body 704 of vessel 701); and to accommodate a motor for motor integration to drive one or more impellers 706 (not seen in this FIG. 7B, but see FIG. 7A). Exemplary ports depicted in FIG. 7B include three liquid-in ports 716 (at 4 o'clock, 6 o'clock and 8 o'clock), one liquid-out port 722 (at 11 'clock), a capacitance sensor 718 (at 9 o'clock), one “gas in” port 724 (at 12 o'clock), one “gas out” port 720 (at 10 o'clock), an optical sensor 726 (at 1 o'clock), a rupture disc 728 (at 2 o'clock), a self-sealing port 730 (at 3 o'clock) to provide access to the main body 704 of growth vessel 701; and a temperature probe 732 (at 5 o'clock).

The ports shown in vessel lid assembly 702 in this FIG. 7B are exemplary only and it should be apparent to one of ordinary skill in the art given the present disclosure that, e.g., a single liquid-in port 716 could be used to accommodate addition of all liquids to the cell culture rather than having a liquid-in port for each different liquid added to the cell culture. Similarly, there may be more than one gas-in port 724, such as one for each gas, e.g., O₂, CO₂ that may be added. In addition, although a temperature probe 732 is shown, a temperature probe alternatively may be located on the outside of vessel holder 714 of bioreactor stand assembly 503 separate from or integrated into heater jacket 748 (not seen in this FIG. 7B, but see FIG. 7E). A self-sealing port 730, if present, allows access to the main body 704 of vessel 701 for, e.g., a pipette, syringe, or other liquid delivery system via a gantry (not shown). As shown in FIG. 7A, additionally there may be a motor integration port to drive the impeller(s), although in other configurations of vessel 701 may alternatively integrate the motor drive at the bottom of the main body 704 of vessel 701. Vessel lid assembly 702 may also comprise a camera port for viewing and monitoring the cells.

Additional sensors include those that detect O₂ concentration, a CO₂ concentration, culture pH, lactate concentration, glucose concentration, biomass, and optical density. The sensors may use optical (e.g., fluorescence detection), electrochemical, or capacitance sensing and either be reusable or configured and fabricated for single-use. Sensors appropriate for use in the bioreactor are available from Omega Engineering (Norwalk Conn.); PreSens Precision Sensing (Regensburg, Germany); C-CIT Sensors AG (Waedenswil, Switzerland), and ABER Instruments Ltd. (Alexandria, Va.). In one embodiment, optical density is measured using a reflective optical density sensor to facilitate sterilization, improve dynamic range and simplify mechanical assembly. The rupture disc, if present, provides safety in a pressurized environment, and is programmed to rupture if a threshold pressure is exceeded in the bioreactor. If the cell culture in the bioreactor vessel is a culture of adherent cells, microcarriers may be used as described supra. In such an instance, the liquid-out port may comprise a filter such as a stainless steel or plastic (e.g., polyvinylidene difluoride (PVDF), nylon, polypropylene, polybutylene, acetal, polyethylene, or polyamide) filter or frit to prevent microcarriers from being drawn out of the culture during, e.g., medium exchange, but to allow dead cells to be withdrawn from the vessel. The microcarriers used for initial cell growth can be nanoporous (where pore sizes are typically <20 nm in size), microporous (with pores between >20 nm to <1 μm in size), or macroporous (with pores between >1 μm in size, e.g. 20 μm) and the microcarriers are typically 50-200 μm in diameter; thus the pore size of the filter or frit in the liquid-out port will differ depending on microcarrier size.

The microcarriers used for cell growth depend on cell type and desired cell numbers, and typically include a coating of a natural or synthetic extracellular matrix or cell adhesion promoters (e.g., antibodies to cell surface proteins or poly-L-lysine) to promote cell growth and adherence. Microcarriers for cell culture are widely commercially available from, e.g., Millipore Sigma, (St. Louis, Mo., USA); ThermoFisher Scientific (Waltham, Mass., USA); Pall Corp. (Port Washington, N.Y., USA); GE Life Sciences (Marlborough, Mass., USA); and Corning Life Sciences (Tewkesbury, Mass., USA). As for the extracellular matrix, natural matrices include collagen, fibrin and vitronectin (available, e.g., from ESBio, Alameda, Calif., USA), and synthetic matrices include MATRIGEL® (Corning Life Sciences, Tewkesbury, Mass., USA), GELTREX™ (ThermoFisher Scientific, Waltham, Mass., USA), CULTREX® (Trevigen, Gaithersburg, Md., USA), biomemetic hydrogels available from Cellendes (Tubingen, Germany); and tissue-specific extracellular matrices available from Xylyx (Brooklyn, N.Y., USA); further, denovoMatrix (Dresden, Germany) offers screenMATRIX™, a tool that facilitates rapid testing of a large variety of cell microenvironments (e.g., extracellular matrices) for optimizing growth of the cells of interest.

FIG. 7C is a side view of the main body 704 of vessel 701. A portion of vessel lid assembly 702 can be seen, as well as two impellers 706a and 706b. Also seen are a lactate/glucose sensor probe 734, a pH, O₂, CO₂ sensor 736 (such as a PRESENS™ integrated optical sensor (Precision Sensing GmbH, (Regensburg, Germany)), and a viable biomass sensor 738 (such as, e.g., the FUTURA PICO™ capacitance sensor (ABER, Alexandria, Va.)). In some embodiments, flat regions are fabricated onto the main body 704 of vessel 701 to reduce optical loss, simplify spot placement and simplify fluorescent measurement of pH, dO₂, and dCO₂.

FIG. 7D shows exemplary design guidelines for a one-impeller embodiment (left) and a two-impeller embodiment (right) of the main body 704 of the vessel, including four exemplary impeller configurations. The embodiment of the INSCRIPTA™ bioreactor vessel 701 main body 704 as shown in this FIG. 7D has a total volume of 820 ml and supports culture volumes from 25 ml to 500 ml. As mentioned above, the impellers (and impeller shaft) may be injection molded or may be fabricated from stainless steel, other biocompatible metals, polymers or plastics and preferably comprised polished surfaces to facilitate sterilization. The impeller may be configured as a turbine-, pitched-blade-, hydrofoil- or marine-type impeller. In a two-impeller configuration, the impellers may be of the same type or different types. In the bioreactors described herein (the “INSCRIPTA™ bioreactors”), agitation is provided at 0-100 rpm, or 40-80 rpm, or approximately 70 rpm during cell growth (depending on the cell type being cultured); however, lower or higher revolutions per minute may be used depending on the volume of the main body 704 of vessel 701, the type of cells being cultured, whether the cells are adherent and being grown on microcarriers or the cells are non-adherent, and the size and configuration of the impellers. The impeller may turn in a clockwise direction, a counter-clockwise direction or the impeller may change direction (oscillate) or stop at desired intervals, particularly during cell detachment from the microcarriers. Also, intermittent agitation may be applied, e.g., agitating for 10 minutes every 30 minutes, or agitating for 1 minute every 5 minutes or any other desired pattern. Additionally, impeller rpm is often increased (e.g., up to 4000 rpm) when the cells are being detached from microcarriers. Although the present embodiment of INSCRIPTA™ bioreactor utilizes one or more impellers for cell growth, alternative embodiments of the INSCRIPTA™ bioreactor described herein may utilize bubbling or other physical mixing means.

Also seen in FIG. 7D is an equation that gives a range for exemplary bioreactor dimensions base on the height (H) and thickness (T) of the main body of vessel 704. For example, D=0.25−05*T means the impeller diameter could be one quarter or one half of the main body of vessel 704 thickness, T. C is the clearance of the impeller from the bottom of the main body of vessel 704, which can be 0.15 to 0.5 times the thickness. It should be apparent to one of ordinary skill in the art given the present disclosure that these numbers are just one embodiment and the ranges may be larger. The bioreactor vessel 701 main body 704 comprises an 8-10 mm clearance from the bottom of the main body 704 of vessel 701 to the lower impeller 706b and the lower impeller 706b and the upper impeller 706a are approximately 40 mm apart.

FIG. 7E is a side view of the vessel holder portion 714 of the bioreactor stand main body 712 of the bioreactor stand assembly 703. Inner surface 740 of vessel holder 714 is indicated and shown are camera or fiber optic ports 746 for monitoring, e.g., cell growth and viability; O₂ and CO₂ levels, and pH. The vessel holder portion 714 of the bioreactor stand main body 712 may also provide illumination using LED lights, such as a ring of LED lights (not shown). FIG. 7F is a side perspective view of the assembled bioreactor without sensors 742. Seen are vessel lid assembly 702, bioreactor stand assembly 703, bioreactor stand main body 712 into which the main body 704 of vessel 701 (not seen in FIG. 7E) is inserted. FIG. 7G is a lower side perspective view of bioreactor assembly 700 showing bioreactor stand assembly 703, bioreactor stand main body 712, vessel lid assembly 702 and two camera mounts 744. Surrounding bioreactor stand main body 712 is heater jacket 748.

FIGS. 7H-1 and 7H-2 together are an exemplary diagram of the bioreactor fluidics. Fluidics and pneumatics are designed to establish a cell culture environment conducive for mammalian cell growth, including iPSCs. Fluidic circuits are designed to deliver and/or remove cell medium, buffers, microcarriers and additional reagents needed for growth, maintenance, selection and passaging of the cells in the automated closed culture instrument. The pneumatic circuits are designed to deliver the appropriate gas mixture and humidity for the chosen cell type, and may comprise line-in filters to prevent any contaminants from reaching the bioreactor.

FIG. 71 is a block diagram for an exemplary bioreactor control system. The control system is designed to control and automate the fluidics, pneumatics and sensor function in a closed system and without human intervention. In one embodiment, the control system is based on state-machines with a user editable state order and parameters using Json and jsonette config files. State-machines allow for dynamic control of several aspects of the bioreactor with a single computer.

In use, the bioreactor described herein is used for cell growth and expansion as well as for medium exchange and cell concentration. Medium/buffer exchange is in one embodiment accomplished using gravitational sedimentation and aspiration via a filter in the liquid-out port where the filter is of an appropriate size to retain microcarriers. In one embodiment used with the present bioreactor, a frit with pore size 100 μm was used and microcarriers with diameters or 120-225 μm were used in the cell culture. Sedimentation was accomplished in approximately 2-3 minutes for a 100 ml culture and 4-5 minutes for a 500 ml culture. The medium was aspirated at >100 ml/min rate. In addition to clearing the medium from the main body 504 of vessel 501, dead cells were removed as well. If sedimentation is used, the microcarriers do not typically accumulate on the filter; however, if accumulation is detected, the medium in the liquid-out port can be pushed back into main body 704 of vessel 701 in a pulse. In some embodiments—particularly those where sedimentation is not used—a cycle of aspiration, release (push back), aspiration and release (push back) may be performed.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example I

### Fully-Automated Singleplex RGN-Directed Editing Run

Singleplex automated genomic editing using MAD7 nuclease was successfully performed with an automated multi-module instrument of the disclosure. See U.S. Pat. No. 9,982,279; and U.S. Ser. No. 16/024,831 filed 30 Jun. 2018; Ser. No. 16/024,816 filed 30 Jun. 2018; Ser. No. 16/147,353 filed 28 Sep. 2018; Ser. No. 16/147,865 filed 30 Sep. 2018; and Ser. No. 16/147,871 filed 30 Jun. 2018.

An ampR plasmid backbone and a lacZ_F172* editing cassette were assembled via Gibson Assembly® into an “editing vector” in an isothermal nucleic acid assembly module included in the automated instrument. lacZ_F172 functionally knocks out the lacZ gene. “lacZ_F172*” indicates that the edit happens at the 172nd residue in the lacZ amino acid sequence. Following assembly, the product was de-salted in the isothermal nucleic acid assembly module using AMPure beads, washed with 80% ethanol, and eluted in buffer. The assembled editing vector and recombineering-ready, electrocompetent E. Coli cells were transferred into a transformation module for electroporation. The cells and nucleic acids were combined and allowed to mix for 1 minute, and electroporation was performed for 30 seconds. The parameters for the poring pulse were: voltage, 2400 V; length, 5 ms; interval, 50 ms; number of pulses, 1; polarity, +. The parameters for the transfer pulses were: Voltage, 150 V; length, 50 ms; interval, 50 ms; number of pulses, 20; polarity, +/−. Following electroporation, the cells were transferred to a recovery module (another growth module), and allowed to recover in SOC medium containing chloramphenicol. Carbenicillin was added to the medium after 1 hour, and the cells were allowed to recover for another 2 hours. After recovery, the cells were held at 4° C. until recovered by the user.

After the automated process and recovery, an aliquot of cells was plated on MacConkey agar base supplemented with lactose (as the sugar substrate), chloramphenicol and carbenicillin and grown until colonies appeared. White colonies represented functionally edited cells, purple colonies represented un-edited cells. All liquid transfers were performed by the automated liquid handling device of the automated multi-module cell processing instrument.

The result of the automated processing was that approximately 1.0E⁰³ total cells were transformed (comparable to conventional benchtop results), and the editing efficiency was 83.5%. The lacZ_172 edit in the white colonies was confirmed by sequencing of the edited region of the genome of the cells. Further, steps of the automated cell processing were observed remotely by webcam and text messages were sent to update the status of the automated processing procedure.

### Example II

### Fully-Automated Recursive Editing Run

Recursive editing was successfully achieved using the automated multi-module cell processing system. An ampR plasmid backbone and a lacZ_V10* editing cassette were assembled via Gibson Assembly® into an “editing vector” in an isothermal nucleic acid assembly module included in the automated system. Similar to the lacZ_F172 edit, the lacZ_V10 edit functionally knocks out the lacZ gene. “lacZ_V10” indicates that the edit happens at amino acid position 10 in the lacZ amino acid sequence. Following assembly, the product was de-salted in the isothermal nucleic acid assembly module using AMPure beads, washed with 80% ethanol, and eluted in buffer. The first assembled editing vector and the recombineering-ready electrocompetent E. Coli cells were transferred into a transformation module for electroporation. The cells and nucleic acids were combined and allowed to mix for 1 minute, and electroporation was performed for 30 seconds. The parameters for the poring pulse were: voltage, 2400 V; length, 5 ms; interval, 50 ms; number of pulses, 1; polarity, +. The parameters for the transfer pulses were: Voltage, 150 V; length, 50 ms; interval, 50 ms; number of pulses, 20; polarity, +/−. Following electroporation, the cells were transferred to a recovery module (another growth module) allowed to recover in SOC medium containing chloramphenicol. Carbenicillin was added to the medium after 1 hour, and the cells were grown for another 2 hours. The cells were then transferred to a centrifuge module and a media exchange was then performed. Cells were resuspended in TB containing chloramphenicol and carbenicillin where the cells were grown to OD600 of 2.7, then concentrated and rendered electrocompetent.

During cell growth, a second editing vector was prepared in an isothermal nucleic acid assembly module. The second editing vector comprised a kanamycin resistance gene, and the editing cassette comprised a galK Y145* edit. If successful, the galK Y145* edit confers on the cells the ability to uptake and metabolize galactose. The edit generated by the galK Y154* cassette introduces a stop codon at the 154th amino acid reside, changing the tyrosine amino acid to a stop codon. This edit makes the galK gene product non-functional and inhibits the cells from being able to metabolize galactose. Following assembly, the second editing vector product was de-salted in the isothermal nucleic acid assembly module using AMPure beads, washed with 80% ethanol, and eluted in buffer. The assembled second editing vector and the electrocompetent E. Coli cells (that were transformed with and selected for the first editing vector) were transferred into a transformation module for electroporation, using the same parameters as detailed above. Following electroporation, the cells were transferred to a recovery module (another growth module), allowed to recover in SOC medium containing carbenicillin. After recovery, the cells were held at 4° C. until retrieved, after which an aliquot of cells were plated on LB agar supplemented with chloramphenicol, and kanamycin. To quantify both lacZ and galK edits, replica patch plates were generated on two media types: 1) MacConkey agar base supplemented with lactose (as the sugar substrate), chloramphenicol, and kanamycin, and 2) MacConkey agar base supplemented with galactose (as the sugar substrate), chloramphenicol, and kanamycin. All liquid transfers were performed by the automated liquid handling device of the automated multi-module cell processing system.

In this recursive editing experiment, 41% of the colonies screened had both the lacZ and galK edits, the results of which were comparable to the double editing efficiencies obtained using a “benchtop” or manual approach.

### Example III

### Ni-NTA Resin Preparation and Regeneration

2 mL IMAC Sepharose 6 FF (GE Healthcare, Chicago, Ill., US) is pipetted into Econo-Pac chromatography columns (Bio-Rad, Hercules, Calif., US), and charged with 15 mL of 100 mM nickel sulfate solution. The charged column is washed with 50 mL of DEMI water and equilibrated with 35 mL of buffer A. After protein purification, columns are regenerated with 10 mL of buffer containing 0.2 M EDTA and 0.5 M NaCl, and washed with 30 mL of 0.5 M NaCl, followed by 30 mL of demineralized water, and stored in 20% ethanol at 4° C.

### Example IV

### Mass Spectrometry

Prior the mass spec analysis, 15 μL of cellular protein extract is subjected to buffer exchange. The samples are diluted to 500 μL in 100 mM ammonium bicarbonate buffer and concentrated by 0.5 mL Amicon Ultra 3 kDa filter unit by centrifugation (14,000 RCF, at 4° C.) to 100 μL. This process is repeated three times with 100 μL of the sample prepared for protease digestion and LC-MS/MS analysis. Samples are submitted to tryptic digestion as follows. First, 90 μL of each sample is denaturated by heating for 10 min at 95° C. Then, disulfide bridges are reduced by incubation with tris(2-carboxyethyl) phosphine at 15 mM final concentration for 1 h at 30° C. Cysteine residues are subsequently alkylated for 30 min with iodoacetamide at 20 mM final concentration at room temperature in the dark. Afterwards protease is added to the reaction mixture in the ratio 1:50 for overnight digestion. The reaction is quenched by addition of triuoroacetic acid to 1% final concentration. Digested samples containing proteolytic peptides are analyzed by LC-MS/MS. 5 μL of each sample is loaded onto a Zorbax Eclipse Plus C18 (1.8 μm, 2.1×150 mm) analytical column from Agilent Technology (Boulder, Colo., US) for separation using analytical Dionex Ultimate 3000 RSLC system from Thermo Scientific (Waltham, Mass., US). The separation is performed with a flow rate of 250 μL/min by applying an effective gradient of solvent B from 5 to 35% in 60 min, followed by column washing and re-equilibration steps. Solvent A is composed of water with 0.1% formic acid, while solvent B consists of acetonitrile with 0.1% formic acid. The outlet of the chromatographic column is coupled online with the conventional HESI source from Thermo Scientific (Waltham, Mass., US) and eluting peptides are analyzed by high resolution QExactive HF-HT-Orbitrap-FT-MS benchtop mass spectrometer from Thermo Scientific (Waltham, Mass., US).

Analysis is performed in a data-dependent manner with 60000 resolution and AGC (automatic gain control) of 3e6 for MS1 scan. MS2 scans are realized in Top10 mode with dynamic exclusion of 30 sec, 15000 resolution, 2 uscans, AGC of 1e5, precursor isolation window of 2 m/z and NCE (normalized collision energy) of 27% for HCD (higher energy collisional dissociation) fragmentation.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

We claim:

1. A method for editing a population of live cells with a library of editing vectors comprising rationally-designed editing cassettes, measuring levels of one or more proteins from the live cells and correlating the edits in the live cells to protein quantities comprising:
designing and synthesizing a library of editing cassettes wherein each editing cassette comprises a gRNA and a repair template, wherein the repair template encodes a barcode construct comprising a peptide barcode, an affinity tag and a protease cleavage site;
inserting the library of editing cassettes into vector backbones to form a library of editing vectors;
transferring the library of editing vectors into a first receptacle;
providing cells to be edited in a second receptacle;
growing the cells to be edited in a growth module;
transferring the cells to be edited from the growth module to a cell concentration module;
concentrating and rendering electrocompetent the cells to be edited in the cell concentration module;
introducing the library of editing vectors into the electrocompetent cells in a transformation module to produce transformed cells;
allowing editing to take place in the transformed cells to produce edited cells, wherein the edited cells transcribe and translate edited proteins comprising the peptide barcode, the affinity tag and the protease cleavage site;
pooling and lysing the edited cells;
performing protease cleavage at the protease cleavage site in the edited proteins to cleave the affinity tag and peptide barcodes from a rest of the edited proteins;
isolating the peptide barcodes via the affinity tags;
identifying and quantitating the peptide barcodes; and
correlating the edits with the quantity of peptide barcodes; wherein the first receptacle, second receptacle, third receptacle, growth module, cell concentration module, transformation module and editing module are all part of a stand-alone automated multi-module cell processing instrument.

2. The method for editing a population of live cells of claim 1, wherein the barcode comprises between four to twenty amino acids.

3. The method for editing a population of live cells of claim 2, wherein the barcode comprises between five to fifteen amino acids.

4. The method for editing a population of live cells of claim 3, wherein the barcode comprises between seven to twelve amino acids.

5. The method for editing a population of live cells of claim 1, wherein the barcode construct is inserted 3′ to a cellular protein start codon and 5′ to the remainder of the coding sequence for the cellular protein.

6. The method for editing a population of live cells of claim 5, wherein the barcode construct comprises 5′ to 3′ the peptide barcode, the affinity tag, and the protease cleavage site.

7. The method for editing a population of live cells of claim 5, wherein the barcode construct comprises 5′ to 3′ the affinity tag, the peptide barcode, and the protease cleavage site.

8. The method for editing a population of live cells of claim 1, wherein the barcode construct is also a DNA barcode which identifies the editing cassette used to create an edit in a cell.

9. The method for editing a population of live cells of claim 1, wherein the barcode construct is inserted 3′ to a cellular protein stop codon.

10. The method for editing a population of live cells of claim 9, wherein the barcode construct comprises 5′ to 3′ the protease cleavage site, the peptide barcode, and the affinity tag.

11. The method for editing a population of live cells of claim 9, wherein the barcode construct comprises 5′ to 3′ the protease cleavage site, the affinity tag, and the peptide barcode.

12. The method for editing a population of live cells of claim 1, wherein the affinity tag is a histidine tag.

13. The method for editing a population of live cells of claim 12, wherein the histidine tag is a His6 tag.

14. The method for editing a population of live cells of claim 1, wherein the affinity tag is a Glutathione S-Transferase tag.

15. The method for editing a population of live cells of claim 1, wherein the affinity tag is a calmodulin-binding tag.

16. The method for editing a population of live cells of claim 1, wherein the protease cleavage site is a TEV protease cleavage site.

17. The method for editing a population of live cells of claim 1, wherein the protease cleavage site is a thrombin protease cleavage site.

18. An editing cassette comprising a gRNA and a repair template, wherein the repair template comprises a coding sequence for a peptide barcode, a coding sequence for an affinity tag, a coding sequence for a protease cleavage site and homology arms complementary to sequences 5′ or 3′ to a cellular protein coding sequence.

19. A library of the editing cassettes of claim 18.

20. The method for editing a population of live cells of claim 1, wherein the barcode construct is separate from a DNA barcode which identifies the editing cassette used to create an edit in a cell.
